(19) United States
(12) Patent Application Publication
(10) Pub. No.: US 2004/0082606 A1
(43) Pub. Date: Apr. 29, 2004

(54) Excitatory amino acid receptor antagonists

(76) Inventors: Vien Van Khau, Carmel, IN (US); Michael Edward Letourneau, Indianapolis, IN (US); Michael John Martinelli, Zionsville, IN (US)

Correspondence Address: ELI LILLY AND COMPANY, PATENT DIVISION, P.O. BOX 6288, INDIANAPOLIS, IN, 46206-6288, US

(21) Appl. No.: 10/450,458

(22) PCT Filed: Dec. 20, 2001
(86) PCT No.: PCT/US01/44715

(51) Int. Cl. A61K 31/5377; A61K 31/4709; C07D 413/14; C 07D 4 3/02

(52) U.S. Cl. 514/310; 546/147; 514/232.8

(57) ABSTRACT

The present invention provides novel pharmaceutically acceptable salts of the compounds of Formula (I) and Formula (Ia), as well as methods for using the pharmaceutically acceptable salts, and also provides processes for making compounds of Formula (I) and Formula (Ia), or the pharmaceutically acceptable salts thereof. Compounds of formula (I) and formula (Ia) are useful for the treatment of neurological disorders, especially migraine.

### BACKGROUND OF THE INVENTION

In the mammalian central nervous system (CNS), the transmission of nerve impulses is controlled by the interaction between a neurotransmitter, that is released by a sending neuron, and a surface receptor on a receiving neuron, which causes excitation of this receiving neuron. L-Glutamate, which is the most abundant neurotransmitter in the CNS, mediates the major excitatory pathways in mammals, and is referred to as an excitatory amino acid (EAA). The receptors that respond to glutamate are called excitatory amino acid receptors (EAA receptors). See Watkins & Evans, Ann. Rev. Pharmacol. Toxicol., 21, 165 (1981); Monaghan, Bridges, and Cotman, Ann. Rev. Pharmacol. Toxicol., 29, 365 (1989); Watkins, Krogsgaard-Larsen, and Honore, Trans. Pharm. Sci., 11, 25 (1990). The excitatory amino acids are of great physiological importance, playing a role in a variety of physiological processes, such as long-term potentiation (learning and memory), the development of synaptic plasticity, motor control, respiration, cardiovascular regulation, and sensory perception.

Excitatory amino acid receptors are classified into two general types. Receptors that are directly coupled to the opening of cation channels in the cell membrane of the neurons are termed “ionotropic.” This type of receptor has been subdivided into at least three subtypes, which are defined by the depolarizing actions of the selective agonists N-methyl-D-aspartate (NMDA), α-amino-3-hydroxy-5-methylisoxazole-4-propionic acid (AMPA), and kainic acid (KA). Molecular biological studies have established that AMPA receptors are composed of subunits (GluR₁-GluR₄), which can assemble to form functional ion channels. Five kainate receptors have been identified which are classified as either High Affinity (KA1 and KA2) or Low Affinity (composed of GluR₅, GluR₆, and/or GluR₇ subunits). Bleakman et al., Molecular Pharmacology, 49, No.4, 581,(1996). The second general type of receptor is the G-protein coupled or second messenger-linked “metabotropic” excitatory amino acid receptor. This second type is coupled to multiple second messenger systems that lead to enhanced phosphoinositide hydrolysis, activation of phospholipase D, increases or decreases in cAMP formation, and changes in ion channel function. Schoepp and Conn, Trends in Pharmacol. Sci., 14, 13 (1993). Both types of excitatory amino acid receptor appear not only to mediate normal synaptic transmission along excitatory pathways, but also to participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

The excessive or inappropriate stimulation of excitatory amino acid receptors leads to neuronal cell damage or loss by way of a mechanism known as excitotoxicity. This process has been suggested to mediate neuronal degeneration in a variety of neurological disorders and conditions. The medical consequences of such neuronal degeneration makes the abatement of these degenerative neurological processes an important therapeutic goal. For instance, excitatory amino acid receptor excitotoxicity has been implicated in the pathophysiology of numerous neurological disorders, including the etiology of cerebral deficits subsequent to cardiac bypass surgery and grafting, stroke, cerebral ischemia, spinal cord lesions resulting from trauma or inflammation, perinatal hypoxia, cardiac arrest, and hypoglycemic neuronal damage. In addition, excitotoxicity has been implicated in chronic neurodegenerative conditions including Alzheimer's Disease, Huntington's Chorea, inherited ataxias, AIDS-induced dementia, amyotrophic lateral sclerosis, idiopathic and drug-induced Parkinson's Disease, as well as ocular damage and retinopathy. Other neurological disorders implicated with excitotoxicity and/or glutamate dysfunction include muscular spasticity including tremors, drug tolerance and withdrawal, brain edema, convulsive disorders including epilepsy, depression, anxiety and anxiety related disorders such as post-traumatic stress syndrome, tardive dyskinesia, and psychosis related to depression, schizophrenia, bipolar disorder, mania, and drug intoxication or addiction. (see generally U.S. Pat. Nos. 5,446,051 and 5,670,516) Excitatory amino acid receptor antagonists may also be useful as analgesic agents and for treating or preventing various forms of headache, including cluster headache, tension-type headache, and chronic daily headache. In addition, published European Patent application WO98/45720 reports that excitatory amino acid receptor excitotoxicity participates in the etiology of acute and chronic pain states including severe pain, intractable pain, neuropathic pain, post-traumatic pain.

It is also known that trigeminal ganglia, and their associated nerve pathways, are associated with painful sensations of the head and face such as headache and, in particular, migraine. Moskowitz (Cephalalgia, 12, 5-7, (1992) proposed that unknown triggers stimulate the trigeminal ganglia which in turn innervate vasculature within cephalic tissue, giving rise to the release of vasoactive neuropeptides from axons innervating the vasculature. These neuropeptides initiate a series of events leading to neurogenic inflammation of the meninges, a consequence of which is pain. This neurogenic inflammation is blocked by sumatriptan at doses similar to those required to treat acute migraine in humans. However, such doses of sumatriptan are associated with contraindications as a result of sumatriptan's attendant vasoconstrictive properties. (see MacIntyre, P. D., et al., British Journal of Clinical Pharmacology, 34, 541-546 (1992); Chester, A. H., et al., Cardiovascular Research, 24, 932-937 (1990); Conner, H. E., et al., European Journal of Pharmacology, 161, 91-94 (1990)). Recently, it has been reported that all five members of the kainate subtype of ionotropic glutamate receptors are expressed on rat trigeminal ganglion neurons, and in particular, high levels of GluR₅ and KA2 have been observed. (Sahara et al., The Journal of Neuroscience, 17(17), 6611 (1997)). As such, migraine presents yet another neurological disorder which may be implicated with glutamate receptor excitotoxicity.

The use of a neuroprotective agent, such as an excitatory amino acid receptor antagonist, is believed to be useful in treating or preventing all of the aforementioned disorders and/or reducing the amount of neurological damage associated with these disorders. For example, studies have shown that AMPA receptor antagonists are neuroprotective in focal and global ischemia models. The competitive AMPA receptor antagonist NBQX (2,3-dihydroxy-6-nitro-7-sulfamoylbenzo[f]quinoxaline) has been reported effective in preventing global and focal ischemic damage. Sheardown et al., Science, 247, 571 (1900); Buchan et al., Neuroreport, 2, 473 (1991); LePeillet et al., Brain Research, 571, 115 (1992). The noncompetitive AMPA receptor antagonists GKYI 52466 has been shown to be an effective neuroprotective agent in rat global ischemia models. LaPeillet et al., Brain Research, 571, 115 (1992). European Patent Application Publication No. 590789A1 and U.S. Pat. No. 5,446,051 and 5,670,516 disclose that certain decahydroisoquinoline derivative compounds are AMPA receptor antagonists and, as such, are useful in the treatment of a multitude of disorders conditions, including pain and migraine headache. WO98/45270 discloses that certain decahydroisoquinoline derivative compounds are selective antagonists of the iGluR₅ receptor and are useful for the treatment of various types of pain, including; severe, chronic, intractable, and neuropathic pain

In accordance with the present invention, Applicants have discovered novel compounds that are selective antagonists of the iGluR₅ receptor subtype and, thus, could be useful in treating the multitude of neurological disorders or neurodegenerative diseases, as discussed above. Such selective antagonists could address a long felt need for safe and effective treatments for neruological disorders, without attending side effects. The treatment of neurological disorders and neurodegenerative diseases is hereby furthered.

### SUMMARY OF THE INVENTION

The present invention provides a compound of Formula I

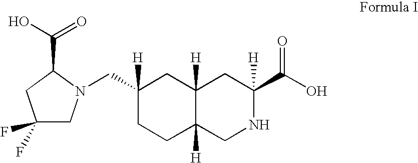

or a pharmaceutically acceptable salt or prodrug thereof.

In a preferred embodiment, the present invention provides a compound of Formula Ia

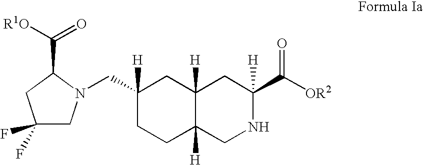

wherein

R¹ and R² each independently represent hydrogen, (C₁-C₂₀)alkyl, (C₂-C₆)alkenyl, (C₁-C₆)alkylaryl, (C₁-C₆)alkyl(C₃-C₁₀)cycloalkyl, (C₁-C₆)alkyl-N,N-C₁-C₆ dialkylamine, (C₁-C₆)alkyl-pyrrolidine, (C₁-C₆)alkyl-piperidine, or (C₁-C₆)alkyl-morpholine, with the proviso that at least one of R¹ and R² are other than hydrogen,

or a pharmaceutically acceptable salt thereof.

In a particularly preferred embodiment, the present invention provides the D-(−)-mandelic acid salt of Formula I or Formula Ia, wherein Formula I and Formula Ia are as defined hereinabove.

In another embodiment, the present invention provides a method of treating or preventing a neurological disorder, or neurodegenerative condition, comprising administering to a patient in need thereof an effective amount of a compound of Formula I or Formula Ia, or a pharmaceutically acceptable salt thereof. Examples of such neurological disorders, or neurodegenerative conditions, include: cerebral deficits subsequent to cardiac bypass surgery and grafting; stroke; cerebral ischemia; spinal cord lesions resulting from trauma or inflammation; perinatal hypoxia; cardiac arrest; hypoglycemic neuronal damage; Alzheimer's Disease; Huntington's Chorea; inherited ataxias; AIDS-induced dementia; amyotrophic lateral sclerosis; idiopathic and drug-induced Parkinson's Disease; ocular damage and retinopathy; muscular spasticity including tremors; drug tolerance and withdrawal; brain edema; convulsive disorders including epilepsy; depression; anxiety and anxiety related disorders such as posttraumatic stress syndrome; tardive dyskinesia; psychosis related to depression, schizophrenia, bipolar disorder, mania, and drug intoxication or addiction; headache, including cluster headache, tension-type headache, and chronic daily headache; migraine; and acute and chronic pain states including severe pain, intractable pain, neuropathic pain, and post-traumatic pain.

Specifically, the present invention provides a method of treating or preventing migraine comprising administering to a patient in need thereof an effective amount of a compound of Formula I or Formula Ia, or a pharmaceutically acceptable salt thereof.

More specifically, the present invention provides a method of treating or preventing migraine comprising administering to a patient in need thereof an effective amount of the D-(−)-mandelic acid salt of Formula I or Formula Ia.

The present invention also provides a process for making a compound of Formula Ia, comprising combining a compound of structure (2)

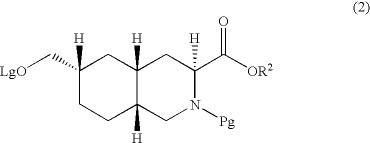

wherein R² is as defined herein, Pg is a suitable nitrogen protecting group, and LgO is a suitable leaving group, with a suitable base in a suitable solvent, followed by addition of a compound of structure (3)

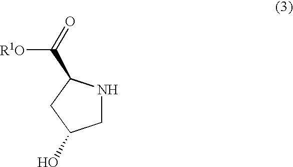

wherein R¹ is as defined herein, followed by oxidation to a compound of structure (5)

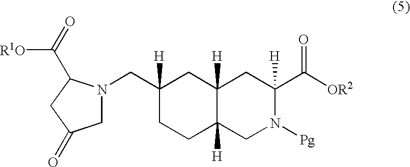

followed by halogenation and removal of the nitrogen protecting group.

In addition, the present invention provides pharmaceutical compositions of compounds of Formula I and Formula Ia, including the pharmaceutically acceptable salts, and hydrates thereof, useful for treating neurological disorders or neurodegenerative conditions, comprising, as an active ingredient, a compound of Formula I or Formula Ia in combination with a pharmaceutically acceptable carrier, diluent or excipient. This invention also encompasses novel intermediates, and processes for the synthesis of the compounds of Formula I and Formula Ia.

More specifically, the present invention provides pharmaceutical compositions useful for treating or preventing migraine comprising, as an active ingredient, the D-(−)-mandelic acid salt of Formula I or Formula Ia, in combination with one or more pharmaceutically acceptable carriers, diluents, or excipients.

The present invention also provides the use of a compound of Formula I or Formula Ia for the manufacture of a medicament for treating or preventing a neurological disorder, or neurodegenerative condition.

More specifically, the present invention provides the use of a compound of Formula I or Formula Ia for the manufacture of a medicament for treating or preventing migraine.

### DETAILED DESCRIPTION OF THE INVENTION

The present invention provides compounds functional as selective iGluR₅ receptor antagonists as well as pharmaceutically acceptable salts, prodrugs, and compositions thereof.

In addition, the present invention provides a method for the treatment of a neurological disorder, or neurodegenerative condition. Particularly, the present invention provides a method for the treatment of migraine which can be demonstrated by a particular mechanism of action, inhibition of neurogenic dural protein extravasation. By treating a migraineur with a compound or composition which is a selective antagonist of the iGluR₅ receptor relative to other excitatory amino acid receptors, the neurogenic extravasation which mediates migraine is inhibited without the attending side effects of agents designed to optimize the 5-HT₁-like mediated vasoconstrictive activity of sumatriptan.

It should be understood by the skilled artisan that all of the compounds useful for the methods of the present invention are available for prodrug formualtion. As used herein, the term “prodrug” refers to a compound of Formula I or which has been structurally modified such that in vivo the prodrug is converted, for example, by hydrolytic, oxidative, reductive, or enzymatic cleavage into the parent compound (e.g. the carboxylic acid (drug), or as the case may be the parent dicarboxylic acid) as given by Formula I. Such prodrugs may be, for example, metabolically labile ester or diester derivatives of the parent compounds having a carboxylic acid group. It is to be understood that the present invention includes any such prodrugs, such as metabolically labile ester or diester derivatives of compounds of the Formula I. In all cases, the use of the compounds described herein as prodrugs is contemplated, and often is preferred, and thus, the prodrugs of all of the compounds employed are encompassed in the names of the compounds herein. Preferred prodrugs include the diester derivatives of Formula I. Conventional procedures for the selection and preparation of suitable prodrugs are well known to one of ordinary skill in the art.

More specifically, examples of prodrugs of Formula I which are understood to be included within the scope of the present invention, are represented by Formula Ia below:

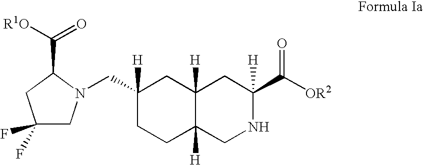

wherein

R¹ and R² each independently represent hydrogen, (C₁-C₂₀)alkyl, (C₂-C₆)alkenyl, (C₁-C₆)alkylaryl, (C₁-C₆)alkyl(C₃-C₁₀)cycloalkyl, (C₁-C₆)alkyl-N,N-C₁-C₆ dialkylamine, (C₁-C₆)alkyl-pyrrolidine, (C₁-C₆)alkyl-piperidine, or (C₁-C₆)alkyl-morpholine, with the proviso that at least one of R¹ and R² are other than hydrogen,

or a pharmaceutically acceptable salt thereof.

It is understood that the selective iGluR₅ receptor antagonists of the present invention may exist as pharmaceutically acceptable salts and, as such, salts are therefore included within the scope of the present invention. The term “pharmaceutically acceptable salt” as used herein, refers to salts of the compounds provided by, or employed in the present invention which are substantially non-toxic to living organisms. Typical pharmaceutically acceptable salts include those salts prepared by reaction of the compounds of the present invention with a pharmaceutically acceptable mineral or organic acid or an organic or inorganic base. Such salts are known as acid addition and base addition salts.

It will be understood by the skilled reader that most or all of the compounds used in the present invention are capable of forming salts, and that the salt forms of pharmaceuticals are commonly used, often because they are more readily crystallized and purified than are the free bases. In all cases, the use of the pharmaceuticals described herein as salts is contemplated in the description herein, and often is preferred, and the pharmaceutically acceptable salts of all of the compounds are included in the names of them.

Acids commonly employed to form acid addition salts are inorganic acids such as hydrochloric acid, hydrobromic acid, hydroiodic acid, sulfuric acid, phosphoric acid, and the like, and organic acids such as p-toluenesulfonic acid, mandelic acid, 1,5-naphthalenedisulfonic acid, methanesulfonic acid, oxalic acid, p-bromophenylsulfonic acid, carbonic acid, succinic acid, citric acid, benzoic acid, acetic acid, and the like. Examples of such pharmaceutically acceptable salts are the sulfate, pyrosulfate, bisulfate, sulfite, bisulfite, phosphate, monohydrogenphosphate, dihydrogenphosphate, metaphosphate, pyrophosphate, bromide, iodide, acetate, propionate, decanoate, caprylate, acrylate, formate, hydrochloride, dihydrochloride, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, hydroxybenzoate, methoxybenzoate, phthalate, xylenesulfonate, phenylacetate, phenylpropionate, phenylbutyrate, citrate, lactate, α-hydroxybutyrate, glycolate, tartrate, methanesulfonate, propanesulfonate, naphthalene-1-sulfonate, napththalene-2-sulfonate, mandelate, napadysilate and the like. Preferred pharmaceutically acceptable acid addition salts are those formed with mineral acids such as hydrochloric acid and hydrobromic acid, and those formed with organic acids such as D-(−)-mandelic acid, 1,5-naphthalenedisulfonic acid, maleic acid, and methanesulfonic acid.

Base addition salts include those derived from inorganic bases, such as ammonium or alkali or alkaline earth metal hydroxides, carbonates, bicarbonates, and the like. Such bases useful in preparing the salts of this invention thus include sodium hydroxide, potassium hydroxide, ammonium hydroxide, potassium carbonate, sodium carbonate, sodium bicarbonate, potassium bicarbonate, calcium hydroxide, calcium carbonate, and the like. The potassium and sodium salt forms are particularly preferred. It should be recognized that the particular counterion forming a part of any salt of this invention is usually not of a critical nature, so long as the salt as a whole is pharmacologically acceptable and as long as the counterion does not contribute undesired qualities to the salt as a whole. It is further understood that such salts may exist as a hydrate.

As used herein, the term “stereoisomer” refers to a compound made up of the same atoms bonded by the same bonds but having different three-dimensional structures which are not interchangeable. The three-dimensional structures are called configurations. As used herein, the term “enantiomer” refers to two stereoisomers whose molecules are nonsuperimposable mirror images of one another. The term “chiral center” refers to a carbon atom to which four different groups are attached. As used herein, the term “diastereomers” refers to stereoisomers which are not enantiomers. In addition, two diastereomers which have a different configuration at only one chiral center are referred to herein as “epimers”. The terms “racemate”, “racemic mixture” or “racemic modification” refer to a mixture of equal parts of enantiomers.

The term “enantiomeric enrichment” as used herein refers to the increase in the amount of one enantiomer as compared to the other. A convenient method of expressing the enantiomeric enrichment achieved is the concept of enantiomeric excess, or “ee”, which is found using the following equation:

$$ee = \frac{{E}^{1} - {E}^{2}}{{E}^{1} + {E}^{2}} \times 100$$

wherein E¹ is the amount of the first enantiomer and E² is the amount of the second enantiomer. Thus, if the initial ratio of the two enantiomers is 50:50, such as is present in a racemic mixture, and an enantiomeric enrichment sufficient to produce a final ratio of 50:30 is achieved, the ee with respect to the first enantiomer is 25%. However, if the final ratio is 90:10, the ee with respect to the first enantiomer is 80%. An ee of greater than 90% is preferred, an ee of greater than 95% is most preferred and an ee of greater than 99% is most especially preferred. Enantiomeric enrichment is readily determined by one of ordinary skill in the art using standard techniques and procedures, such as gas or high performance liquid chromatography with a chiral column. Choice of the appropriate chiral column, eluent and conditions necessary to effect separation of the enantiomeric pair is well within the knowledge of one of ordinary skill in the art. In addition, the enantiomers of compounds of Formula I or Formula Ia can be resolved by one of ordinary skill in the art using standard techniques well known in the art, such as those described by J. Jacques, et al., “Enantiomers, Racemates, and Resolutions”, John Wiley and Sons, Inc., 1981.

The compounds of the present invention have one or more chiral centers and may exist in a variety of stereoisomeric configurations. As a consequence of these chiral centers, the compounds of the present invention occur as racemates, mixtures of enantiomers and as individual enantiomers, as well as diastereomers and mixtures of diastereomers. All such racemates, enantiomers, and diastereomers are within the scope of the present invention.

The terms “R” and “S” are used herein as commonly used in organic chemistry to denote specific configuration of a chiral center. The term “R” (rectus) refers to that configuration of a chiral center with a clockwise relationship of group priorities (highest to second lowest) when viewed along the bond toward the lowest priority group. The term “S” (sinister) refers to that configuration of a chiral center with a counterclockwise relationship of group priorities (highest to second lowest) when viewed along the bond toward the lowest priority group. The priority of groups is based upon their atomic number (in order of decreasing atomic number). A partial list of priorities and a discussion of stereochemistry is contained in “Nomenclature of Organic Compounds: Principles and Practice”, (J. H. Fletcher, et al., eds., 1974) at pages 103-120.

The specific stereoisomers and enantiomers of compounds of Formula I and Formula Ia can be prepared by one of ordinary skill in the art utilizing well known techniques and processes, such as those disclosed by Eliel and Wilen, “Stereochemistry of Organic Compounds”, John Wiley & Sons, Inc., 1994, Chapter 7, Separation of Stereoisomers. Resolution. Racemization, and by Collet and Wilen, “Enantiomers, Racemates, and Resolutions”, John Wiley & Sons, Inc., 1981. For example, the specific stereoisomers and enantiomers can be prepared by stereospecific syntheses using enantiomerically and geometrically pure, or enantiomerically or geometrically enriched starting materials. In addition, the specific stereoisomers and enantiomers can be resolved and recovered by techniques such as chromatography on chiral stationary phases, enzymatic resolution or fractional recrystallization of addition salts formed by reagents used for that purpose.

As used herein the term “Pg” refers to a suitable nitrogen protecting group.

Examples of a suitable nitrogen protecting group as used herein refers to those groups intended to protect or block the nitrogen group against undesirable reactions during synthetic procedures. Choice of the suitable nitrogen protecting group used will depend upon the conditions that will be employed in subsequent reaction steps wherein protection is required, and is well within the knowledge of one of ordinary skill in the art. Commonly used nitrogen protecting groups are disclosed in Greene, “Protective Groups In Organic Synthesis,” (John Wiley & Sons, New York (1981)). Suitable nitrogen protecting groups comprise acyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, .alpha.-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, .alpha.,.alpha.dimethyl-3,5-dimethoxybenzyloxycarbonyl, benzhydryloxycarbonyl, t-butyloxycarbonyl, diisopropylmethoxycarbonyl, isopropyloxycarbonyl, ethoxycarbonyl, methoxycarbonyl, allyloxycarbonyl, 2,2,2,-trichloroethoxycarbonyl, phenoxycarbonyl, 4-nitrophenoxycarbonyl, fluorenyl-9-methoxycarbonyl, cyclopentyloxycarbonyl, adamantyloxycarbonyl, cyclohexyloxycarbonyl, phenylthiocarbonyl and the like; alkyl groups such as benzyl, triphenylmethyl, benzyloxymethyl and the like; and silyl groups such as trimethylsilyl and the like. Preferred suitable nitrogen protecting groups are formyl, acetyl, methoxycarbonyl, benzoyl, pivaloyl, t-butylacetyl, phenylsulfonyl, benzyl, t-butyloxycarbonyl (Boc) and benzyloxycarbonyl (Cbz).

As used herein the term “(C₁-C₄)alkyl” refers to a straight or branched, monovalent, saturated aliphatic chain of 1 to 4 carbon atoms and includes, but is not limited to methyl, ethyl, n-propyl, isopropyl, n-butyl, isobutyl and the like.

As used herein the term “(C₁-C₆)alkyl” refers to a straight or branched, monovalent, saturated aliphatic chain of 1 to 6 carbon atoms and includes, but is not limited to methyl, ethyl, n-propyl, isopropyl, n-butyl, isobutyl, t-butyl, n-pentyl, n-hexyl, and the like.

As used herein the term “(C₁-C₁₀)alkyl” refers to a straight or branched, monovalent, saturated aliphatic chain of 1 to 10 carbon atoms and includes, but is not limited to methyl, ethyl, propyl, isopropyl, n-butyl, isobutyl, tertiary butyl, pentyl, isopentyl, hexyl, 2,3-dimethyl-2-butyl, heptyl, 2,2-dimethyl-3-pentyl, 2-methyl-2-hexyl, octyl, 4-methyl-3-heptyl and the like.

As used herein the term “(C₁-C₂₀)alkyl” refers to a straight or branched, monovalent, saturated aliphatic chain of 1 to 20 carbon atoms and includes, but is not limited to, methyl, ethyl, propyl, isopropyl, butyl, isobutyl, t-butyl, pentyl, isopentyl, hexyl, 3-methylpentyl, 2-ethylbutyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-nonadecyl, n-eicosyl and the like. It is understood that the terms “(C₁-C₄)alkyl”, “(C₁-C₆)alkyl”, and “(C₁-C₁₀)alkyl” are included within the definition of “(C₁-C₂₀)alkyl”.

As used herein, the terms “Me”, “Et”, “Pr”, “iPr”, “Bu” and “t-Bu” refer to methyl, ethyl, propyl, isopropyl, butyl and tert-butyl respectively.

As used herein, the term “(C₁-C₄)alkoxy” refers to an oxygen atom bearing a straight or branched, monovalent, saturated aliphatic chain of 1 to 4 carbon atoms and includes, but is not limited to methyoxy, ethyoxy, n-propoxy, isopropoxy, n-butoxy, and the like.

As used herein the term “(C₁-C₆)alkoxy” refers to an oxygen atom bearing a straight or branched, monovalent, saturated aliphatic chain of 1 to 6 carbon atoms and includes, but is not limited to methoxy, ethoxy, n-propoxy, isopropoxy, n-butoxy, n-pentoxy, n-hexoxy, and the like.

As used herein, the term “(C₁-C₆)alkyl(C₁-C₆)alkoxy” refers to a straight or branched, monovalent, saturated aliphatic chain of 1 to 6 carbon atoms which has a (C₁-C₆)alkoxy group attached to the aliphatic chain.

As used herein, the terms “Halo”, “Halide” or “Hal” refer to a chlorine, bromine, iodine or fluorine atom, unless otherwise specified herein. As used herein the term “(C₂-C₆)alkenyl” refers to a straight or branched, monovalent, unsaturated aliphatic chain having from two to six carbon atoms. Typical C₂-C₆ alkenyl groups include ethenyl (also known as vinyl), 1-methylethenyl, 1-methyl-1-propenyl, 1-butenyl, 1-hexenyl, 2-methyl-2-propenyl, 1-propenyl, 2-propenyl, 2-butenyl, 2-pentenyl, and the like.

As used herein, the term “aryl” refers to a monovalent carbocyclic group containing one or more fused or non-fused phenyl rings and includes, for example, phenyl, 1- or 2-naphthyl, 1,2-dihydronaphthyl, 1,2,3,4-tetrahydronaphthyl, and the like. The term “substituted aryl” refers to an aryl group substituted with one or two moieties chosen from the group consisting of halogen, hydroxy, cyano, nitro, (C₁-C₆)alkyl, (C₁C₄)alkoxy, (C₁-C₆)alkyl(C₃-C₁₀)cycloalkyl, (C₁-C₆)alkylaryl, (C₁-C₆)alkoxycarbonyl, protected carboxy, carboxymethyl, hydroxymethyl, amino, aminomethyl, or trifluoromethyl.

As used herein, the term “(C₁-C₆)alkylaryl” refers to a straight or branched, monovalent, saturated aliphatic chain of 1 to 6 carbon atoms which has an aryl group attached to the aliphatic chain. Included within the term “C₁-C₆ alkylaryl” are the following:

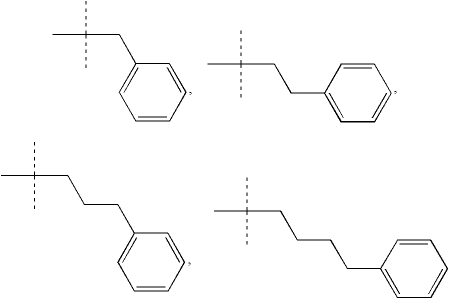

and the like.

As used herein, the term “aryl(C₁-C₆)alkyl” refers to an aryl group which has a straight or branched, monovalent, saturated aliphatic chain of 1 to 6 carbon atoms attached to the aryl group. Included within the term “aryl(C₁-C₆)alkyl” are the following:

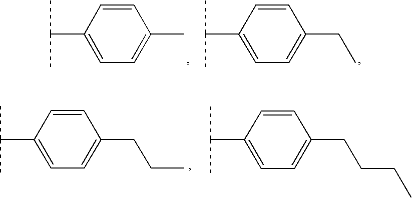

and the like.

As used herein the term “(C₃-C₁₀)cycloalkyl” refers to a saturated hydrocarbon ring structure composed of one or more fused or unfused rings containing from three to ten carbon atoms. Typical C₃-C₁₀ cycloalkyl groups include cyclopropyl, cyclobutyl, cyclopentyl, cyclohexyl, cycloheptyl, cyclooctyl, adamantanyl, and the like.

As used herein, the term “C₁-C₆ alkyl(C₃-C₁₀)cycloalkyl” refers to a straight or branched, monovalent, saturated aliphatic chain of 1 to 6 carbon atoms which has a (C₃-C₁₀)cycloalkyl attached to the aliphatic chain. Included within the term “C₁-C₆ alkyl(C₃-C₁₀)cycloalkyl” are the following:

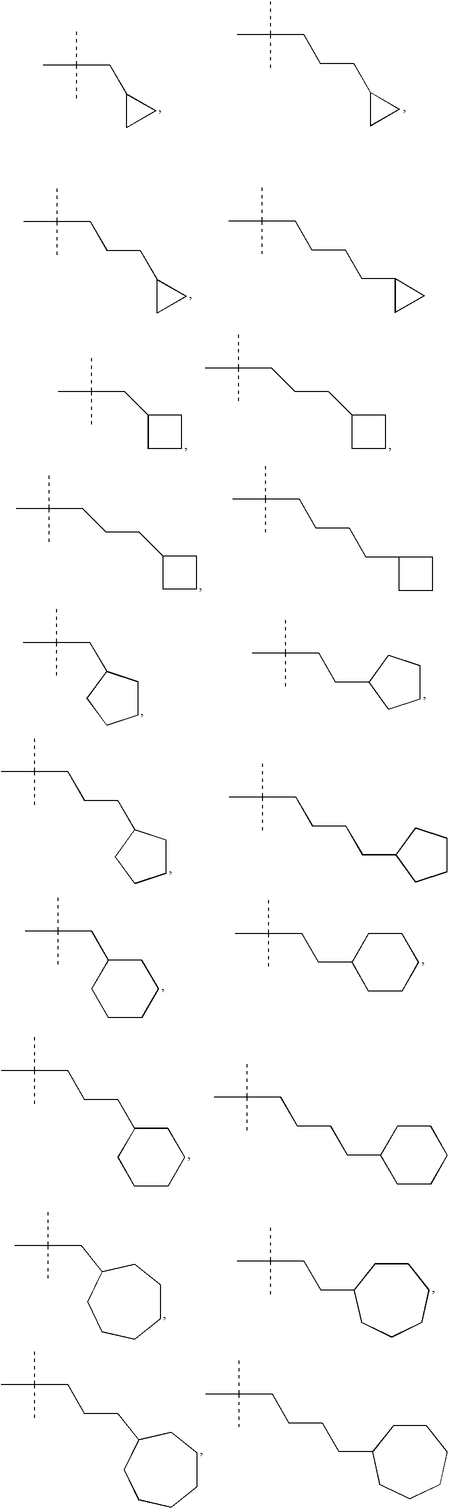

and the like.

As used herein, the term “(C₁-C₆) alkoxycarbonyl” refers to a carbonyl group having a (C₁-C₆)alkyl group attached to the carbonyl carbon through an oxygen atom. Examples of this group include t-buoxycarbonyl, methoxycarbonyl, and the like.

As used herein the term “heterocycle” refers to a five- or six-membered ring, which contains one to four heteroatoms selected from the group consisting of oxygen, sulfur, and nitorgen. The remaining atoms of the ring are recognized as carbon by those of skill in the art. Rings may be saturated or unsaturated. Examples of heterocycle groups include thiophenyl, furyl, pyrrolyl, imidazolyl, pyrrazolyl, thiazolyl, thiazolidinyl, isothiazolyl, oxazolyl, isoxazolyl, triazolyl, thiadiazolyl, oxadiazolyl, tetrazolyl, pyridyl, pyrimidyl, pyrazinyl, pyridiazinyl, triazinyl, imidazolyl, dihydropyrimidyl, tetrahydropyrimdyl, pyrrolidinyl, piperidinyl, piperazinyl, pyrazolidinyl, pyrimidinyl, imidazolidimyl, morpholinyl, pyranyl, thiomorpholinyl, and the like. The term “substituted heterocycle” represents a heterocycle group substituted with one or two moieties chosen from the group consisting of halogen, hydroxy, cyano, nitro, oxo, (C₁-C₆)alkyl, (C₁-C₄)alkoxy, C₁-C₆ alkyl(C₃-C₁₀)cycloalkyl, (C₁-C₆)alkylaryl, (C₁-C₆)alkoxycarbonyl, protected carboxy, carboxymethyl, hydroxymethyl, amino, aminomethyl, or trifluoromethyl.

As used herein the term “N,N-C₁-C₆ dialkylamine” refers to a nitrogen atom substituted with two straight or branched, monovalent, saturated aliphatic chains of 1 to 6 carbon atoms. Included within the term “N,N-C₁-C₆ dialkylamine” are —N(CH₃)₂, N(CH₂CH₃)₂, —N(CH₂CH₂CH₃)₂, —N(CH₂CH₂CH₂CH₃)₂, and the like.

As used herein the term “C₁-C₆alkyl-N,N-C₁-C₆dialkylamine” refers to straight or branched, monovalent, saturated aliphatic chain of 1 to 6 carbon atoms which has an N,N-C₁-C₆ dialkylamine attached to the aliphatic chain. Included within the term “C₁-C₆ alkyl-N,N-C₁-C₆ dialkylamine” are the following:

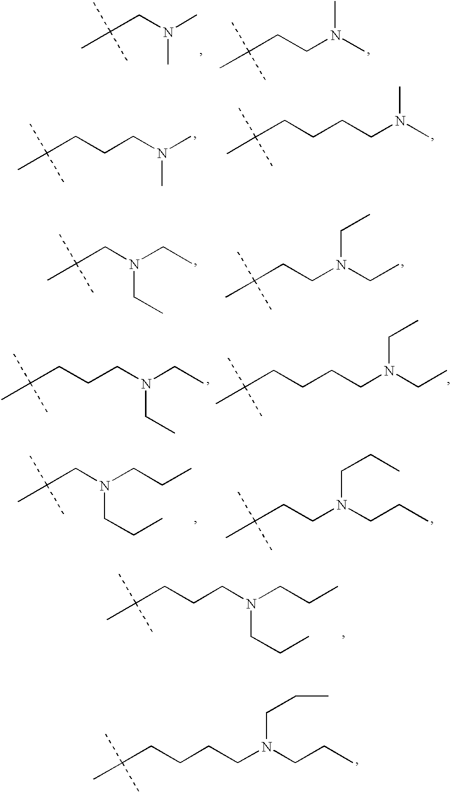

and the like.

As used herein the term “(C₁-C₆)alkyl-pyrrolidine” refers to a straight or branched, monovalent, saturated aliphatic chain of 1 to 6 carbon atoms which has a pyrrolidine attached to the aliphatic chain. Included within the scope of the term “(C₁-C₆)alkyl-pyrrolidine” are the following:

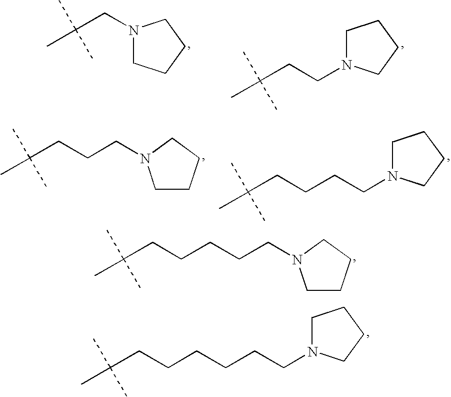

and the like.

As used herein the term “(C₁-C₆)alkyl-piperidine” refers to a straight or branched, monovalent, saturated aliphatic chain of 1 to 6 carbon atoms which has a piperidine attached to the aliphatic chain. Included within the scope of the term “(C₁-C₆)alkyl-piperidine” are the following:

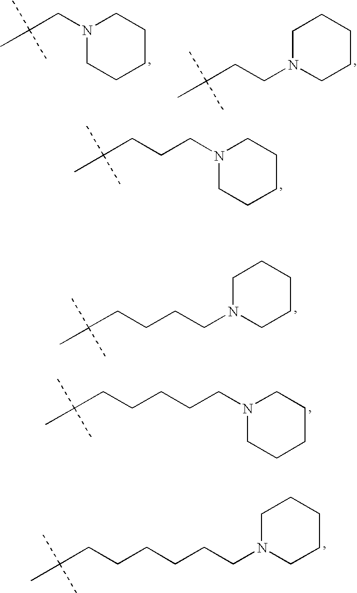

and the like.

As used herein the term “(C₁-C₆)alkyl-morpholine” refers to a straight or branched, monovalent, saturated aliphatic chain of 1 to 6 carbon atoms which has a morpholine attached to the aliphatic chain. Included within the scope of the term “C₁-C₆ alkyl-morpholine” are the following:

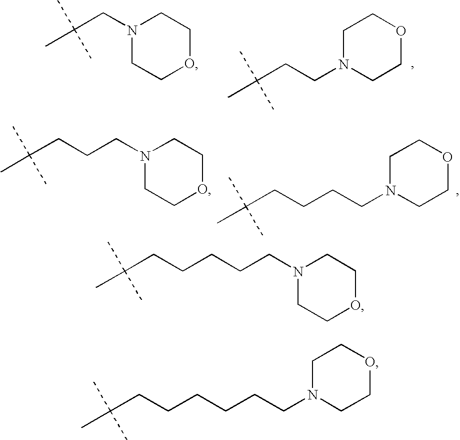

and the like.

The designation “ [symbol] ” refers to a bond that protrudes forward out of the plane of the page.

The designation “ [symbol] ” refers to a bond that protrudes backward out of the plane of the page.

As used herein the term “iGluR₅” refers to the kainate ionotropic glutamate receptor, subtype 5, of the larger class of excitatory amino acid receptors.

As used herein the term “migraine” refers a disorder of the nervous system characterized by recurrent attacks of head pain (which are not caused by a structural brain abnormalitiy such as those resulting from tumor or stroke), gasrointestinal disturbances, and possibly neurological symptoms such as visual distortion. Characteristic headaches of migraine usually last one day and are commonly accompanied by nausea, emesis, and photophobia.

Migraine may represent a “chronic” condition, or an “acute” episode. The term “chronic”, as used herein, means a condition of slow progress and long continuance. As such, a chronic condition is treated when it is diagnosed and treatment continued throughout the course of the disease. Conversely, the term “acute” means an exacerbated event or attack, of short course, followed by a period of remission. Thus, the treatment of migraine contemplates both acute events and chronic conditions. In an acute event, compound is administered at the onset of symptoms and discontinued when the symptoms disappear. As described above, a chronic condition is treated throughout the course of the disease.

As used herein the term “patient” refers to a mammal, such a mouse, gerbil, guinea pig, rat, dog or human. It is understood, however, that the preferred patient is a human.

It is understood that the term “selective iGluR₅ receptor antagonist” as used herein, includes those excitatory amino acid receptor antagonists which selectively bind to the iGluR₅ kainate receptor subtype, relative to the iGluR₂ AMPA receptor subtype. Preferably the selective iGluR₅ antagonist for use according to the methods of the present invention has a binding affinity at least 10 fold greater for iGluR₅ than for iGluR₂, more preferably at least 100 fold greater. Selective iGluR₅ receptor antagonists are readily available to, or are readily prepared by, one of ordinary skill in the art following recognized procedures. For example, WO 98/45270 provides examples of selective iGluR₅ receptor antagonists and discloses methods for synthesis.

As used herein, the terms “treating”, “treatment”, or “to treat” each mean to alleviate symptoms, eliminate the causation of resultant symptoms either on a temporary or permanent basis, and to prevent, slow the appearance, or reverse the progression or severity of resultant symptoms of the named disorder. As such, the methods of this invention encompass both therapeutic and prophylactic administration.

As used herein the term “effective amount” refers to the amount or dose of the compound, upon single or multiple dose administration to the patient, which provides the desired effect in the patient under diagnosis or treatment. An effective amount can be readily determined by the attending diagnostician, as one skilled in the art, by the use of known techniques and by observing results obtained under analogous circumstances. In determining the effective amount or dose of compound administered, a number of factors are considered by the attending diagnostician, including, but not limited to: the species of mammal; its size, age, and general health; the degree of involvement or the severity of the migraine involved; the response of the individual patient; the particular compound administered; the mode of administration; the bioavailability characteristics of the preparation administered; the dose regimen selected; the use of concomitant medication; and other relevant circumstances.

A typical daily dose will contain from about 0.01 mg/kg to about 100 mg/kg of each compound used in the present method of treatment. Preferably, daily doses will be about 0.05 mg/kg to about 50 mg/kg, more preferably from about 0.1 mg/kg to about 25 mg/kg.

Oral administration is a preferred route of administering the compounds employed in the present invention whether administered alone, or as a combination of compounds capable of acting as a selective iGluR₅ receptor antagonist. Oral administration, however, is not the only route, nor even the only preferred route. Other preferred routes of administration include transdermal, percutaneous, intravenous, intramuscular, intranasal, buccal, or intrarectal routes. Where the selective iGluR₅ receptor antagonist is administered as a combination of compounds, one of the compounds may be administered by one route, such as oral, and the other may be administered by the transdermal, percutaneous, intravenous, intramuscular, intranasal, pulmonary, buccal, or intrarectal route, as particular circumstances require. The route of administration may be varied in any way, limited by the physical properties of the compounds and the convenience of the patient and the caregiver.

The compounds employed in the present invention may be administered as pharmaceutical compositions and, therefore, pharmaceutical compositions incorporating compounds of Formula I or Formula Ia are important embodiments of the present invention. Such compositions may take any physical form that is pharmaceutically acceptable, but orally administered pharmaceutical compositions are particularly preferred. Such pharmaceutical compositions contain, as an active ingredient, an effective amount of a compound of Formula I or Formula Ia, including the pharmaceutically acceptable salts, prodrugs, and hydrates thereof, which effective amount is related to the daily dose of the compound to be administered. Each dosage unit may contain the daily dose of a given compound, or may contain a fraction of the daily dose, such as one-half or one-third of the dose. The amount of each compound to be contained in each dosage unit depends on the identity of the particular compound chosen for the therapy, and other factors such as the indication for which it is given. The pharmaceutical compositions of the present invention may be formulated so as to provide quick, sustained, or delayed release of the active ingredient after administration to the patient by employing well known procedures.

Compositions are preferably formulated in a unit dosage form, each dosage containing from about 1 to about 500 mg of each compound individually or in a single unit dosage form, more preferably about 5 to about 300 mg (for example 25 mg). The term “unit dosage form” refers to a physically discrete unit suitable as unitary dosages for a patient, each unit containing a predetermined quantity of active material calculated to produce the desired therapeutic effect, in association with a suitable pharmaceutical carrier, diluent, or excipient.

The inert ingredients and manner of formulation of the pharmaceutical compositions are conventional. The usual methods of formulation used in pharmaceutical science may be used here. All of the usual types of compositions may be used, including tablets, chewable tablets, capsules, solutions, parenteral solutions, intranasal sprays or powders, troches, suppositories, transdermal patches and suspensions. In general, compositions contain from about 0.5% to about 50% of the compounds in total, depending on the desired doses and the type of composition to be used. The amount of the compound, however, is best defined as the “effective amount”, that is, the amount of each compound which provides the desired dose to the patient in need of such treatment.

The activity of the compounds employed in the present invention do not depend on the nature of the composition, hence, the compositions are chosen and formulated solely for convenience and economy.

Capsules are prepared by mixing the compound with a suitable diluent and filling the proper amount of the mixture in capsules. The usual diluents include inert powdered substances such as starches, powdered cellulose especially crystalline and microcrystalline cellulose, sugars such as fructose, mannitol and sucrose, grain flours, and similar edible powders.

Tablets are prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

Tablets are often coated with sugar as a flavor and sealant. The compounds may also be formulated as chewable tablets, by using large amounts of pleasant-tasting substances such as mannitol in the formulation, as is now well-established practice.

Instantly dissolving tablet-like formulations are also now frequently used to assure that the patient consumes the dosage form, and to avoid the difficulty in swallowing solid objects that bothers some patients.

A lubricant is often necessary in a tablet formulation to prevent the tablet and punches from sticking in the die. The lubricant is chosen from such slippery solids as talc, magnesium and calcium stearate, stearic acid and hydrogenated vegetable oils.

Tablet disintegrators are substances which swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethylcellulose, for example, may be used, as well as sodium lauryl sulfate.

Enteric formulations are often used to protect an active ingredient from the strongly acid contents of the stomach. Such formulations are created by coating a solid dosage form with a film of a polymer which is insoluble in acid environments, and soluble in basic environments. Exemplary films are cellulose acetate phthalate, polyvinyl acetate phthalate, hydroxypropyl methylcellulose phthalate and hydroxypropyl methylcellulose acetate succinate.

When it is desired to administer the compound as a suppository, the usual bases may be used. Cocoa butter is a traditional suppository base, which may be modified by addition of waxes to raise its melting point slightly. Water-miscible suppository bases comprising, particularly, polyethylene glycols of various molecular weights are in wide use, also.

Transdermal patches have become popular recently. Typically they comprise a resinous composition in which the drugs will dissolve, or partially dissolve, which is held in contact with the skin by a film which protects the composition. Many patents have appeared in the field recently. Other, more complicated patch compositions are also in use, particularly those having a membrane pierced with innumerable pores through which the drugs are pumped by osmotic action.

The following table provides an illustrative list of formulations suitable for use with the compounds employed in the present invention. The following is provided only to illustrate the invention and should not be interpreted as limiting the present invention in any way.

### Formulation 1

Hard gelatin capsules are prepared using the following ingredients:

| | Quantity | |
|---|---|---|
| | (mg/capsule) | |
| Active Ingredient | 250 | |
| Starch, dried | 200 | |
| Magnesium stearate | 10 | |
| Total | 460 | mg |

The above ingredients are mixed and filled into hard gelatin capsules in 460 mg quantities.

### Formulation 2

A tablet is prepared using the ingredients below:

| | Quantity | |
|---|---|---|
| | (mg/tablet) | |
| Active Ingredient | 250 | |
| Cellulose, microcrystalline | 400 | |
| Silicon dioxide, fumed | 10 | |
| Stearic acid | 5 | |
| Total | 665 | mg |

The components are blended and compressed to form tablets each weighing 665 mg.

### Formulation 3

An aerosol solution is prepared containing the following components:

| | Weight % |
|---|---|
| Active Ingredient | 0.25 |
| Ethanol | 29.75 |
| Propellant 22 | 70.00 |
| (Chlorodifluoromethane) | |
| Total | 100.00 |

The active compound is mixed with ethanol and the mixture added to a portion of the Propellant 22, cooled to −30° C. and transferred to a filling device. The required amount is then fed to a stainless steel container and diluted with the remainder of the propellant. The valve units are then fitted to the container.

### Formulation 4

Tablets each containing 60 mg of active ingredient are made as follows:

| | | |
|---|---|---|
| Active Ingredient | 60.0 | mg |
| Starch | 45.0 | mg |
| Microcrystalline cellulose | 35.0 | mg |
| Polyvinylpyrrolidone | 4.0 | mg |
| Sodium carboxymethyl starch | 4.5 | mg |
| Magnesium stearate | 0.5 | mg |
| Talc | 1.0 | mg |
| Total | 150 | mg |

The active ingredient, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50° C. and passed through a No. 18 mesh U.S. sieve. The sodium carboxymethyl starch, magnesium stearate, and talc, previously passed through a No. 60 mesh U.S. sieve, are then added to the granules which, after mixing, are compressed on a tablet machine to yield tablets each weighing 150 mg.

### Formulation 5

Capsules each containing 80 mg medicament are made as follows:

| | | |
|---|---|---|
| Active Ingredient | 80 | mg |
| Starch | 59 | mg |
| Microcrystalline cellulose | 59 | mg |
| Magnesium stearate | 2 | mg |
| Total | 200 | mg |

The active ingredient, cellulose, starch, and magnesium stearate are blended, passed through a No. 45 sieve, and filled into hard gelatin capsules in 200 mg quantities.

### Formulation 6

Suppositories each containing 225 mg of active ingredient may be made as follows:

| | | |
|---|---|---|
| Active Ingredient | 225 | mg |
| Saturated fatty acid glycerides | 2,000 | mg |
| Total | 2,225 | mg |

The active ingredient is passed through a No. 60 mesh U.S. sieve and suspended in the saturated fatty acid glycerides previously melted using the minimum heat necessary.

The mixture is then poured into a suppository mold of nominal 2 g capacity and cooled.

### Formulation 7

Suspensions each containing 50 mg of medicament per 5 ml dose are made as follows:

| Active Ingredient | 50 | mg |
|---|---|---|
| Sodium carboxymethyl cellulose | 50 | mg |
| Syrup | 1.25 | ml |
| Benzoic acid solution | 0.10 | ml |
| Flavor | q.v. | |
| Color | q.v. | |
| Purified water to total | 5 | ml |

The medicament is passed through a No. 45 mesh U.S. sieve and mixed with the sodium carboxymethyl cellulose and syrup to form a smooth paste. The benzoic acid solution, flavor and color are diluted with some of the water and added, with stirring. Sufficient water is then added to produce the required volume.

### Formulation 8

An intravenous formulation may be prepared as follows:

| | | |
|---|---|---|
| Active Ingredient | 100 | mg |
| Mannitol | 100 | mg |
| 5 N Sodium hydroxide | 200 | ml |
| Purified water to total | 5 | ml |

It is understood by one of ordinary skill in the art that the procedures as described above can also be readily applied to a method of treating neurological disorders or neurodegenerative conditions, particularly migraine, comprising administering to a patient an effective amount of a compound of Formula I or Formula Ia.

Compounds of Formula I and Formula Ia can be prepared, for example, by following the procedures set forth in Scheme I. These schemes are not intended to limit the scope of the invention in any way. All substituents, unless otherwise indicated, are previously defined. The reagents and starting materials are readily available to one of ordinary skill in the art. For example, certain necessary starting materials can be prepared by one of ordinary skill in the art following procedures disclosed in U.S. Pat. No. 5,356,902 (issued Oct. 18, 1994) and U.S. Pat. No. 5,446,051 (issued Aug. 29, 1995).

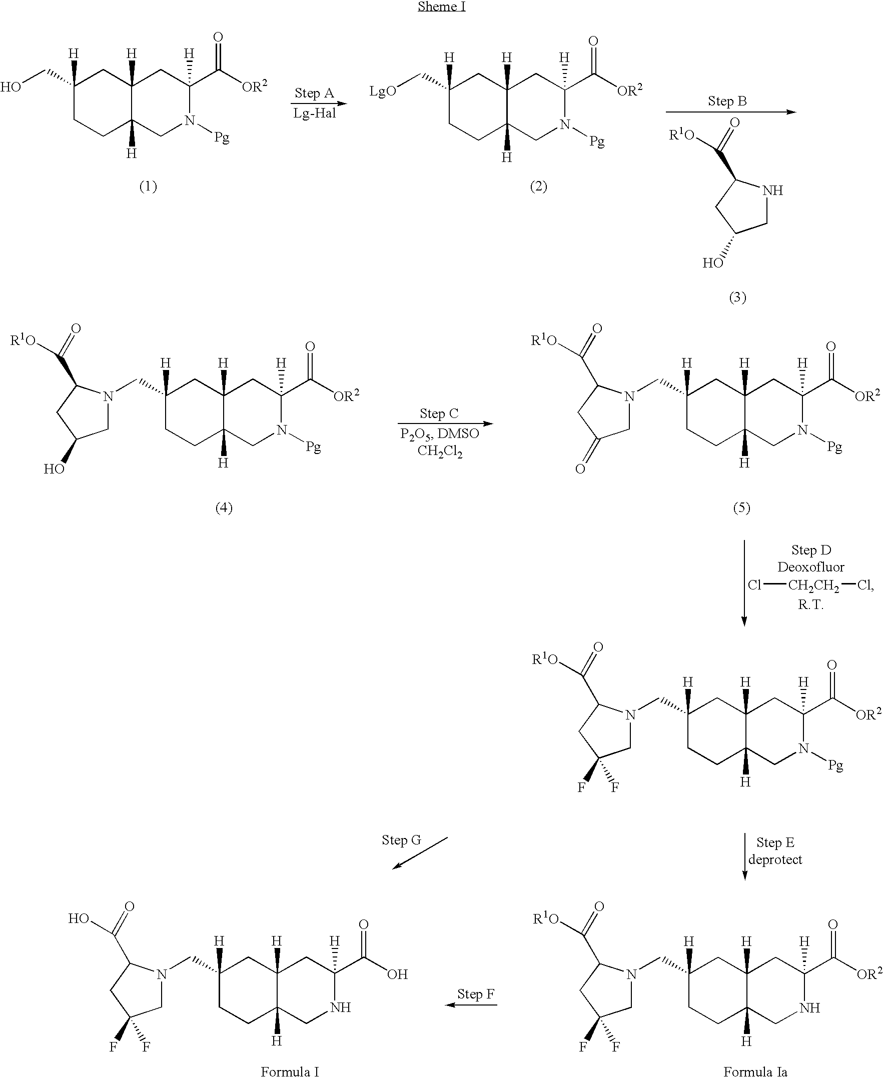

In Scheme I, step A, the 6-(hydroxymethyl)-2-(methoxycarbonyl)-decahydroisoquinoline-3-carboxylate of compound (1) (wherein Pg is a suitable nitrogen protecting group as defined hereinabove, with methoxycarbonyl being preferred) is treated under standard conditions with a compound of formula Lg-Hal, wherein Lg is a suitable leaving group and Hal represents a chloro, bromo or iodo atom, to provide the compound of structure (2). For example, a solution of compound (1), dissolved in a suitable organic solvent such as dichloromethane and cooled to 0° C., is treated with an excess of a suitable organic base, such as triethylamine, followed by about 1 to 2 equivalents of a compound of formula Lg-Hal. Examples of Lg-Hal include m-nitrobenzenesulfonyl chloride, p10 nitrobenzenesulfonyl chloride, p-bromobenzenesulfonyl chloride, p-toluenesulfonyl chloride, benzenesulfonyl chloride, methanesulfonyl chloride, trifluoromethanesulfonyl chloride, and the like an addition, one skilled in the art will appreciate that a halo atom itself, such as chloro, bromo, or iodo may also be used as a suitable leaving group in place of LgO.) The reaction mixture is warmed to room temperature and stirred for about 5 to 20 hours. The compound (2) is then isolated using standard procedures. For example, the reaction mixture is washed with water, the organic layer separated and dried over anhydrous sodium sulfate, filtered, and concentrated under vacuum to provide crude compound (2). Column chromatography is then performed with on silica gel with a suitable eluent such as 10-50% ethyl acetate/hexane to provide the purified compound (2).

In Scheme I, Step 13, compound (2) is treated under standard conditions with a pyrrolidine of structure (3) to provide the compound of structure (4). For example, compound (2) is mixed with about 1-1.5 equivalents of 4-hydroxy-L-proline ethyl ester (R³ is ethyl) and 1-1.5 equivalents of potassium carbonate and heated at reflux in a suitable solvent such as acetonitrile for about 60-70 hours. The reaction mixture is cooled to room temperature and solvents removed under vacuum. Compound (4) is then isolated using standard procedures such as extraction techniques. For example, the reaction mixture is partitioned between water and an organic solvent such as diethyl ether, and the aqueous layer extracted 2-6 times with diethyl ether. The organic layers are combined, dried over anhydrous sodium sulfate, filtered, and concentrated under vacuum to provide compound (4). Compound (4) can then be purified by chromatography on silica gel with a suitable eluent such as 10-50% ethyl acetate/hexanes or methanol/chloroform.

Alternatively in Step B, a pyrrolidine of structure (3) may be combined with a suitable resin filter cake and the resulting mixture treated with the compound of structure (2) to provide the compound of structure (4). For example, Amberlite IRA 67 resin is treated with water, then stirred and filtered and the filter cake washed with a suitable solvent such as acetone until the water content of the wash is less than about 5% by weight. The filter cake is combined with 4-hydroxy-L-proline ethyl ester hydrochloride and acetone and the mixture stirred at RT for about 1-2 hrs. The mixture is then filtered and the filter cake washed again with acetone. This filtered solution of hydroxyproline ethyl ester may then be used directly. A slurry of Amberlite IRA 67 resin in water is stirred and the mixture filtered. The filter cake is washed with a suitable solvent such as acetone until the water content of the wash was less than about 5% by weight. This second filter cake may then be combined with the hydroxyproline ethyl ester solution from above and a solution of compound (2), and the mixture heated at reflux. After about 24 hours, the reaction is cooled and filtered. The filter cake is washed with dichloromethane (about 300 mL) and the combined filtrates are concentrated in vacuo. The residue may then be concentrated from dichloromethane several times to provide compound (4) as an oil to be used directly in the next step.

In Scheme I, Step C, compound (4) is oxidized under standard conditions to give the compound of structure (5). For example, a solution of dichloromethane is treated with phosphorus pentoxide an the reaction allowed to cool to about −10° C. Dimethyl sulfoxide is added with stirring and the reaction mixture is then treated with a solution of compound (4) dissolved in dichloromethane. The reaction is warmed to about 20-22° C. over a period of about 4-5 hours, stirred for about 8-20 hours, cooled to about 0° C. then treated with triethylamine at such a rate so as to maintain the reaction temperature below about 5° C. The reaction is allowed to warm to R.T., stirred for about one hour, and then added to a solution of 0.1M HCl at such a rate so as to maintain the reaction temperature below about 10° C. The compound (5) is then isolated using standard procedures. For example, additional dichloromethane is added to partition the reaction mixture. The aqueous layer is then extracted 2-6 times with dichloromethane and the organics combined, washed with 1M NaHCO₃, dried over magnesium sulfate, then concentrated under vacuum to provide compound (5). The crude material may then be purified by chromatography on silica gel with a suitable eluent such as 10-50% ethyl acetate in toluene or methanol/dichoromethane.

In Scheme I, Step D, compound (5) is flourinated to provide the compound of structure (6). For example, a solution of compound (5) in ethanol is dissolved in a suitable organic solvent such as 1,2-dichloroethane, treated with Deoxofluor ([Bis-(2-methoxyethyl)amino]sulfur trifluoride) and stirred at R.T for about 20-25 hours. The reaction mixture is then treated with a concentrated solution of NaHCO₃ and stirred for about 15 minutes. The layers are separated and the aqueous layer extracted 2-6 times with toluene. The layers are separated and the organics combined, filtered, dried over Na₂SO₄, and then concentrated under vacuum to provide the compound of structure (6). The crude material may then be purified by chromatography on silica gel with a sutiable eluent such as (50:50)toluene/heptane and or 10-50% ethyl acetate in toluene.

In Scheme I, Step E, the compound of structure (6) is deprotected under standard conditions well known in the art to provide the compound of Formula Ia. For example, when Pg is a methoxycarbonyl protecting group, compound (6) is dissolved in a suitable organic solvent such as dichloromethane under an atmosphere of nitrogen and treated with trimethylsilyl iodide. The reation mixture is allowed to warm to room temperature and stirred for 10-20 hours. The reaction is quenched by addition of saturated aqueous NaHCO₃. The aqueous layer is then extracted 2-6 times with dichloromethane. The organics are then combined, washed with a 1N solution of sodium thiosulfate, dried over magnesium sulfate, filtered, and concentrated under vacuum to provide the compound of Formula Ia. The material may then be purified by chromatography on silica gel with a suitable eluent such as methanol/dichoromethane, to provide the purified compound of Formula Ia.

In Scheme I, Step F, the compound of Formula Ia may be optionally hydrolyzed to the compound of Formula I under conditions well known in the art. For example, the compound of Formula Ia is dissolved in a suitable organic solvent such as methanol, and treated with an excess of a suitable base. Examples of suitable bases include aqueous lithium hydroxide, sodium hydroxide, potassium hydroxide, and the like with lithium hydroxide being preferred. The reaction is stirred for about 10-20 hours. The reaction mixture is then neutralized to pH 6 with 1N HCl and concentrated under vacuum to provide the crude compound of Formula I. This material may then be then purified by techniques well known in the art, such as chromatography with a suitable eluent.

In Scheme I, Step G, compound (6) optionally may be concomitantly deprotected and hydrolyzed to provide the compound of Formula I. For example, a solution of compound (6) dissolved in 6.0N HCl is heated at reflux for about 15-20 hours. The reaction mixture is then allowed to cool to room temperature and concentrated under vacuum to provide the compound of Formula I. The compound of Formula I may then purified by techniques well known in the art, such as cation exchange chromatography eluting with methanol/water followed by 2 N ammonia in methanol or ethanol to provide the purified compound of Formula I.

In addition, one skilled in the art will recognize that the compound of Formula I can be esterified under standard conditions to provide the compound of Formula Ia. For example, the compound of Formula I may be dissolved in a suitable organic solvent such as ethanol, and treated with an excess of a suitable acid. Examples of suitable acids include gaseous hydrochloric acid, aqueous sulfuric acid, p-toluene sulfonic acid, and the like with gaseous hydrochloric acid being a preferred acid. The reaction mixture is heated to reflux for a suitable time. The reaction mixture may then be concentrated, for example, under vacuum to provide the crude compound, of Formula Ia. This material may then be purified by techniques well known in the art, such as cation exchange chromatography eluting with methanol/water followed by 2 N ammonia in ethanol to provide the purified compound of Formula Ia.

The Formula I and Formula Ia compounds of the present invention may be chemically synthesized from a common intermediate, 6-hydroxymethyl-2-methoxycarbonyl-decahydroisoquinoline-3-carboxylate. This intermediate, in turn, may be chemically synthesized from a 6-oxo-2-methoxycarbonyl-decahydroisoquinoline-3-carboxylic acid intermediate, the synthesis of which is described in U.S. Pat. No. 4,902,695, No. 5,446,051, and No. 5,356,902 (the entire contents of which are all herein incorporated by reference.)

Routes for the synthesis of the 6-(hydroxymethyl)-2-(methoxycarbonyl)-decahydroisoquinoline-3-carboxylate intermediate, useful for the synthesis of the compounds of the present invention, are shown in Schemes IIa and IIb below.

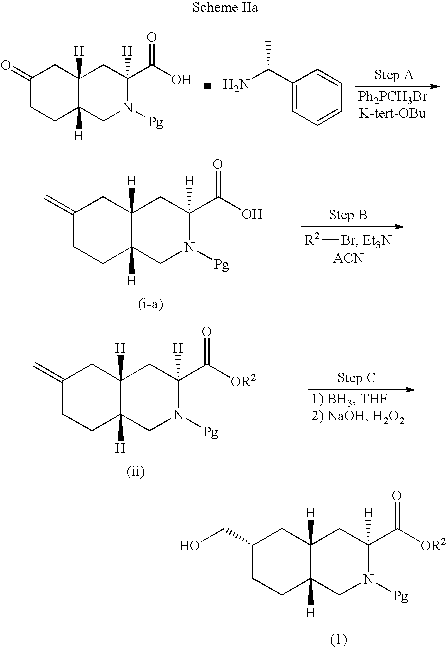

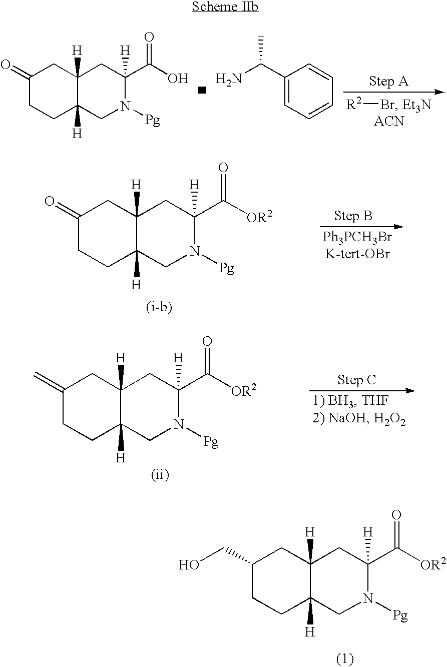

In Scheme IIa, Step A, 6-oxo-decahydroisoquinoline-3-carboxylic acid is treated with methyltriphenylphosphonium bromide to provide the 6-methylidine-decahydroisoquinoline-3-carboxylic acid of compound (i-a). For example, a slurry of 1 equivalent of 6-oxo-2-methoxycarbonyl-decahydroisoquinoline-3-carboxylic acid and about 1.4 equivalents of methyltriphenylphosphonium bromide in THF and DMF is stirred mechanically under an atmosphere of nitrogen and cooled to −10° C. Potassium tert-butoxide solution (2.4 equiv in THF) is added dropwise over a 10 minute period. The slurry is allowed to warm to room temperature and stirred thus for 2.5 hours (complete by TLC at this time). The reaction is partitioned between water and EtOAc and the layers are separated. The organic phase is extracted 2 times with water and the aqueous portions are combined and washed 2-6 times with dichloromethane. The aqueous solution is made acidic by addition of 6 M HCl solution and extracted 2-6 times with dichloromethane. These last three organic extracts are combined, dried with sodium sulfate and concentrated under reduced pressure to provide the compound of structure (i-a).

In Scheme IIa, Step B, the intermediate 6-methylidine-decahydroisoquinoline-3-carboxylic acid (compound (i-a)) is esterified by reaction with a compound of formula R²—Br (where R² is as herein defined) to provide the 6-methylidine-decahydroisoquinoline-3-carboxylate intermediate of compound (ii). For example 6-methylidine-2-methoxycarbonyl-decahydroisoquinoline-3-carboxylic acid is dissolved in acetonitrile and treated with triethylamine and bromoethane. The reaction is heated at 50° C. for about 3 hours, cooled and partitioned between 50:50 ethyl acetate/heptane and 1N HCL. The organic phase is isolated and washed 3 times with water, saturated sodium bicarbonate, brine, dried over anhydrous sodium sulfate, filtered, and concentrated under vacuum to provide the compound of structure (ii). This crude material is dissolved in 10% ethyl acetate/heptane and applied to a plug of silica gel (10 g in 10% ethyl acetate/heptane). The plug is eluted with, 10% ethyl acetate/heptane, 15% ethyl acetate/heptane, and 25% ethyl acetate/heptane. The eluents are combined and concentrated under vacuum to provide the purified compound of structure (ii).

In Scheme IIa, Step C, the 6-methylidine-decahydroisoquinoline-3-carboxylate intermediate (compound (ii)) is subjected to hydroboration, followed by oxidation to provide the 6-hydroxymethyl-decahydroisoquinoline-3-carboxylate intermediate of compound (1). For example, Ethyl-6-methylidine-2-methoxycarbonyl-decahydroisoquinoline-3-carboxylate is dissolved in THF and cooled to about −15° C. under an atmosphere of nitrogen with stirring. A 1M solution of BH₃.THF is added dropwise over 5-7 minutes and the reaction mixture is stirred for about 2 hours at −10 to −12° C. The reaction is then slowly treated with a suitable base, such as lithium or sodium hydroxide, and then treated slowly with 30% H₂O₂ over 15 minutes. The reaction mixture is allowed to warm to room temperature and then partitioned between ethyl acetate and 50% saturated sodium chloride solution. The aqueous layer is extracted with ethyl acetate and the combined organics are washed with sodium bisulfite solution, brine, dried over anhydrous sodium sulfate, filtered, and concentrated under vacuum to provide the intermediate of compound (1).

Alternatively, the 6-hydroxymethyl-2-methoxycarbonyl-decahydroisoquinoline-3-carboxylate intermediate (compund (1)) may be made according to the synthetic route described in Scheme IIb. In Scheme IIb, Step A, 6-oxo-decahydroisoquinoline-3-carboxylic acid is esterified by reaction with a compound of formula R²—Br (where R² is as herein defined) to provide the 6-oxo-decahydroisoquinoline-3-carboxylate intermediate of compound (i-b). For example 6-oxo-2-methoxycarbonyl-decahydroisoquinoline-3-carboxylic acid is dissolved in acetonitrile and treated with tiethylamine and bromoethane. The reaction is heated at 50° C. for about 3 hours, cooled and partitioned between 50:50 ethyl acetate/heptane and 1N HCL. The organic phase is isolated and washed 3 times with water, saturated sodium bicarbonate, brine, dried over anhydrous sodium sulfate, filtered, and concentrated under vacuum to provide the compound of structure (i-b). This crude material is dissolved in 10% ethyl acetate/heptane and applied to a plug of silica gel (10 g in 10% ethyl acetate/heptane). The plug is eluted with, 10% ethyl acetate/heptane, 15% ethyl acetate/heptane, and 25% ethyl acetate/heptane. The eluents are combined and concentrated under vacuum to provide the purified compound of structure (i-b).

In Scheme IIb, Step B, the 6-oxo-decahydroisoquinoline-3-carboxylate intermediate of compound (i-b) is treated with methyltriphenylphosphonium bromide to provide the 6-methylidine-decahydroisoquinoline-3-carboxylate of compound (ii). For example a slurry of 1 equivalent of 6-oxo-2-methoxycarbonyl-decahydroisoquinoline-3-carboxylate (compound (i-b)) and about 1.4 equivalents of methyltriphenylphosphonium bromide in THF and DMF is stirred mechanically under an atmosphere of nitrogen and cooled to −10° C. Potassium tert-butoxide solution (2.4 equiv in THF) is added dropwise over a 10 minute period. The slurry is allowed to warm to room temperature and stirred thus for 2.5 hours (complete by TLC at this time). The reaction is partitioned between water and EtOAc and the layers are separated. The organic phase is extracted 2 times with water and the aqueous portions are combined and washed 2-6 times with dichloromethane. The aqueous solution is made acidic by addition of 6 M HCl solution and extracted 2-6 times with dichloromethane. These last three organic extracts are combined, dried with sodium sulfate and concentrated under reduced pressure to provide the compound of structure (ii).

In Scheme IIb, Step C, following the procedures as described in Scheme IIa, Step C above, the 6-methylidine-decahydroisoquinoline-3-carboxylate intermediate (compound (ii)) is subjected to hydroboration, followed by oxidation to provide the 6-hydroxymethyl-decahydroisoquinoline-3-carboxylate intermediate of compound (1).

The following preparations and examples illustrate the compounds and methods of the present invention. The reagents and starting materials are readily available to one of ordinary skill in the art. These examples are intended to be illustrative only and are not to be construed so as to limit the scope of the invention in any way. As used herein, the following terms have the meanings indicated: “i.v.” refers to intravenously; “p.o.” refers to orally; “i.p.” refers to intraperitoneally; “eq” or “equiv.” refers to equivalents; “g” refers to grams; “mg” refers to milligrams; “L” refers to liters; “mL” refers to milliliters; “μL” refers to microliters; “mol” refers to moles; “mmol” refers to millimoles; “psi” refers to pounds per square inch; “mm Hg” refers to millimeters of mercury; “min” refers to minutes; “h” or “hr” refers to hours; “° C.” refers to degrees Celsius; “TLC” refers to thin layer chromatography; “HPLC” refers to high performance liquid chromatography; “R_{f}” refers to retention factor; “Rₜ” refers to retention time; “δ” refers to part per million down-field from tetramethylsilane; “THF” refers to tetrahydrofuran; “DMF” refers to N,N-dimethylformamide; “DMSO” refers to dimethyl sulfoxide; “aq” refers to aqueous, “EtOAc” refers to ethyl acetate; “iPrOAc” refers to isopropyl acetate; “MeOH” refers to methanol; “MTBE” refers to tert-butyl methyl ether; “RT” refers to room temperature; “Kᵢ” refers to the dissociation constant of an enzyme-antagonist complex and serves as an index of ligand binding; and “ID₅₀” and “ID₁₀₀” refer to doses of an administered therapeutic agent which produce, respectively, a 50% and 100% reduction in a physiological response.

### Preparation 1

### Preparation of [3S,4aR,6S,8aR]-6-methylidine-2-(methoxycarbonyl)-1,2,3,4,4a,5,6,7,8,8a-decahydroisoquinoline-3-carboxylic Acid

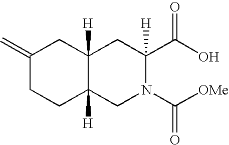

A slurry of methyltriphenylphosphonium bromide (12.4 g, 34.6 mmol) in THF (25 mL) is cooled to −10 to −12° C. under an atmosphere of nitrogen and treated with sodium hexamethyldisilazide (35 mL of a 1M solution in THF) via syringe over 6 to 8 minutes with stirring. The reaction mixture is then stirred for 20 minutes at −10 to −12° C. and added via cannula over 3-4 minutes to [3S,4aR,6S,8aR]-6-oxo-2-(methoxycarbonyl)-1,2,3,4,4a,5,6,7,8,8a-decahydroisoquinoline-3-carboxylic acid (10.0 g, 26.6 mmol), [which had previously been treated with sodium hexamethyldisilazide (27 mL of 1 M solution in THF) at 0-3° C. and stirred for 10 minutes] dissolved in DMF (20 mL) and cooled to 0-3° C. under an atmosphere of nitrogen. This is followed by a THF (3 mL) rinse of the flask holding the Wittig reagent which is also added to the reaction mixture. The reaction mixture is then allowed to stir for 5 minutes at 0-3° C., then allowed to warm to room temperature, and stirred for an additional 3 hours. Ethyl acetate (100 ML) and water (50 mL) are added with stirring and then the layers are separated. The organic layer is extracted with water (50 mL) and the combined aqueous portions are washed with methylene chloride (5×75 mL). The aqueous is then treated with 6M HCl (15 mL) and extracted with methylene chloride (3×50 mL). The organic extracts are dried over anhydrous sodium sulfate, filtered, and concentrated under vacuum to provide the title compound (6.59 g, 98%) as a yellow oil.

### Alternative Synthesis of [3S,4aR,6S,8aR]-6-methylidine-2-(methoxycarbonyl)-1,2,3,4,4a,5,6,7,8,8a-decahydroisoquinoline-3-carboxylic Acid

A slurry of [3S,4aR,6S,8aR]-6-oxo-2-(methoxycarbonyl)-1,2,3,4,4a,5,6,7,8,8a-decahydroisoquinoline-3-carboxylic acid (50.0 g, 0.133 mol, 1.0 equiv) and methyltriphenylphosphonium bromide (66.4 g, 0.186 mol, 1.4 equiv) in THF (150 mL) and DMF (25 mL) is stirred mechanically under an atmosphere of nitrogen and cooled to −10° C. Potassium tert-butoxide solution (187 mL of 1.7 M in THF, 0.319 mol, 2.4 equiv) is added dropwise over a 10 minute period. A mild exotherm throughout this addition results in an increase of the reaction temperature to 6° C. The slurry is allowed to warm to room temperature and stirred thus for 2.5 hours (complete by TLC at this time). Reaction is partitioned between water (250 mL) and EtOAc (250 mL) and the layers are separated. The organic phase is extracted with water (2×100 mL) and the aqueous portions are combined and washed with dichloromethane (5×300 mL). The aqueous solution is made acidic by addition of 6 M HCl solution (50 mL) and extracted with dichloromethane (3×150 mL). These last three organic extracts are combined, dried with sodium sulfate and concentrated under reduced pressure to provide the title compound as a yellow film (36.17 g). Estimated potency of the product by proton NMR is 89 wt % (remainder residual solvents) for a corrected yield of 32.2 g (95.6%).

### Preparation 2

### Preparation of [3S,4aR,6S,8aR]-Ethyl-6-methylidine-2-(methoxycarbonyl)-1,2,3,4,4a,5,6,7,8,8a-decahydroisoquinoline-3-carboxylate.

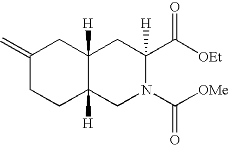

[3S,4aR,6S,8aR]-6-methylidine-2-(methoxycarbonyl)-1,2,3,4,4a,5,6,7,8,8a-decahydroisoquinoline-3-carboxylic acid (6.59 g, 26.0 mmol, prepared in preparation 1) is dissolved in acetonitrile (26 mL) and treated with triethylamine (7.25 mL, 52 mmol) and bromoethane (5.82 mL, 78 mmol). The reaction is heated at 50° C. for about 3 hours, cooled and partitioned between 50:50 ethyl acetate/heptane (100 ml) and 1N HCL (75 mL). The organic phase is isolated and washed with water (3×30 mL), saturated sodium bicarbonate (30 mL), brine (30 mL), dried over anhydrous sodium sulfate, filtered, and concentrated under vacuum to provide the crude title compound as an amber oil. This crude material is dissolved in 10% ethyl acetate/heptane (15 mL) and applied to a plug of silica gel (10 g in 10% ethyl acetate/heptane). The plug is eluted with 10% ethyl acetate/heptane (10 mL), 15% ethyl acetate/heptane (15 mL), and 25% ethyl acetate/heptane (90 mL). The eluents are combined and concentrated under vacuum to provide the purified titled compound (6.84 g, 91%) as a colorless oil.

### Preparation 3

### Preparation of [3S,4aR,6S,8aR]-Ethyl-6-(hydroxymethyl)-2-(methoxycarbonyl)-1,2,3,4,4a,5,6,7,8,8a-decahydroisoquinoline-3-carboxylate.

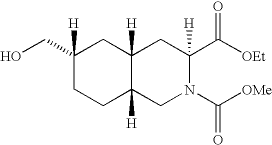

[3S,4aR,6S,8aR]-Ethyl-6-methylidine-2-(methoxycarbonyl)-1,2,3,4,4a,5,6,7,8,8a-decahydroisoquinoline-3-carboxylate (2.0 g, 7.11 mmol, prepared in preparation 2) is dissolved in THF (10 mL) and heptane (2 mL) and cooled to about −15° C. under an atmosphere of nitrogen with stirring. A 1M solution of BH₃.THF in THF (3.91 mL, 3.91 mmol) is added dropwise over 5-7 minutes and the reaction is stirred for about 2-4 hours at −10 to −14° C. The reaction is treated with ethanol (1.25 mL) over 2 minutes then allowed to warm to 20° C. The reaction is then treated slowly with 1M LiOH solution (3.91 mL), followed by slow addition of 30% H₂O₂ (1.2 mL addition at such a rate that the reaction temperature remains below 30° C.). The reaction mixture is allowed stir at room temperature for 30-45 minutes and then partitioned between ethyl acetate (12 mL) and 10% sodium bisulfite solution (14 mL). The organic layer is washed with 10% sodium bisulfite solution (16 mL), brine (8 mL), dried over anhydrous sodium sulfate, filtered, and concentrated under vacuum to provide the title compound (2.01 g) as a colorless oil.

### EXAMPLE 1

### 3S,4aR,6S,8aR Ethyl 6-(((2S)-2-(Ethoxycarbonyl)-4,4-difluoropyrrolidinyl)methyl)-1,2,3,4,4a,5,6,7,8,8a-decahydroisoquinoline-3-carboxylate.D-(−)-mandelic Acid

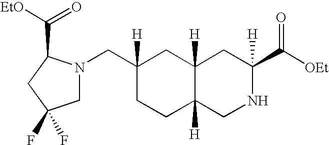

A. Preparation of

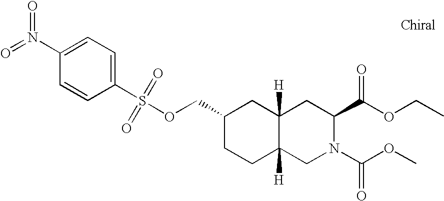

A solution of [3S,4aR,6S,8aR]-Ethyl-6-(hydroxymethyl)-2-(methoxycarbonyl)1,2,3,4,4a,5,6,7,8,8a-decahydroisoquinoline-3-carboxylate (37.5 g, 0.125 mol) and triethylamine (35.0 mL, 0.25 mol) in EtOAc (94 mL) is added dropwise to a solution of p-nitrobenzenesulfonyl chloride (28.6 g, 0.125 mol) in EtOAc (94 mL) maintained at 0-2° C. (addition time: 30.min.). Reaction is allowed to warm to RT and stirred for about 2.5 hr then quenched by addition of water (100 mL), 1M HCl (100 mL) and brine (20 mL). The layers are separated and the organic phase washed with 1M NaHCO₃ solution (150 mL), brine (150 mL) and dried over MgSO₄. Concentration in vacuo provides the title compound as an oil (58.9 g, 97%). ¹H NMR (CDCl₃) δ8.41 (2H, d), 8.05 (2H, d), 4.38 (t, 1H), 4.17 (m, 2H), 3.97 m, 2H), 3.69 (s, 3H), 3.39 (m, 2H), 2.12 (m, 1H), 1.85 (m, 3H), 1.55 (m, 5H), 1.25 (m, 5H).

B. Preparation of

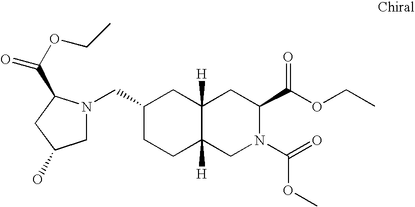

Amberlite IRA 67 resin (334.7 g) is treated with water (about 680 mL) and stirred. The mixture is filtered and the filter cake is washed with acetone until the water content of the wash is less than 5% by weight. This filter cake is combined with hydroxyproline ethyl ester HCl, 72.7 g, 0.372 mol) and acetone (700 mL) and the mixture stirred at RT for about 1-2 hrs. The mixture is filtered and the filter cake is washed with acetone (300 mL). This filtered solution of hydroxyproline ethyl ester is used directly. A slurry of Amberlite IRA 67 resin (465 g) in water (about 900 mL) is stirred and then the mixture is filtered. The filter cake is washed with acetone until the water content of the wash is less than 5% by weight. This filter cake is combined with the hydroxyproline ethyl ester solution from above and a solution of the compound from Step A above (100 g, 0.206 mol) in EtOAc (about 150 mL) and the mixture is heated at reflux. After about 24 hours, the reaction is cooled and filtered. The filter cake is washed with dichloromethane (about 300 mL) and the combined filtrates are concentrated in vacuo and the residue is concentrated from dichloromethane several times to provide the title compound as an oil to be used directly in the next step.

C. Preparation of

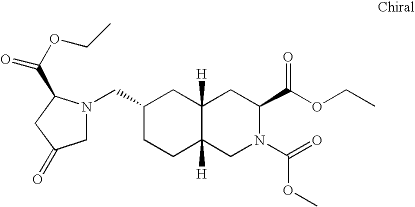

Dichloromethane (about 230 mL) is treated with phosphorous pentoxide (117.2 g, 0.825 mol) and the mixture is cooled to about −10° C. Dimethyl sulfoxide (96.8 g, 1.24 mol) is added to the mixture at such a rate that the reaction temperature remains at about −10° C. The reaction is stirred at about −10° C. and treated with a solution of the compound of Step B above in dichloromethane (about 230 mL) at such a rate that the reaction temperature remains below about −10° C. The reaction is warmed to about 20-22° C. over a few hours (4-5) and stirred overnight then cooled to about 0° C. and treated with triethylamine (83.5 g, 0.825 mol) at such a rate that the reaction temperature remains below 5° C. The reaction is allowed to warm to RT and stirred for 1 hour. The reaction is added to a 0.1M HCl solution (about 460 mL pre-cooled to about 0-5° C.) at such a rate that the reaction temperature remains below 10° C. Additional dichloromethane (about 460 mL) is added and the mixture is stirred briefly and the layers separated. The aqueous portion is extracted with dichloromethane (about 460 mL) and the combined organic extracts are washed with 1M NaHCO₃ (about 460 mL). The layers are separated and the organic layer is dried over magnesium sulfate and concentrated in vacuo. The crude product is purified by chromatography using Silica Gel 60 and 35% EtOAc in toluene to provide the title compound as an oil (36.9 g, 41% from 504476). ¹H NMR (CDCl₃) δ4.39 (t, 1H), 4.18 (m, 4H), 3.72 (m, 1H), 3.70 (s, 3H), 3.40 (m, 3H), 3.00 (d, 1H), 2.40-2.69 (m, 4H), 2.08 (m, 1H), 1.45-1.95 (m, 9H), 1.30 (m, 6H), 1.10 (m, 1H).

D. Preparation of

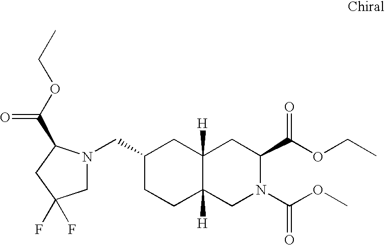

A solution of the compound from Step C above (8.98 g, 0.0205 mol) and ethanol (0.227 μL, 0.0039 mol) in 1,2-dichloroethane (43 mL) is treated with Deoxofluor ([Bis-5 (2-methoxyethyl)amino]sulfur trifluoride, 6.5 mL, 0.0353 mol) and the reaction is stirred at RT for about 21 hours. The reaction is treated with a saturated solution of NaHCO₃ in water (60 mL) and the mixture is then stirred for 15 min. The layers are separated and the aqueous portion is extracted with toluene (about 45 mL). The layers are separated and the toluene and 1,2-dicloroethane solutions combined and dried over Na₂SO₄. The drying agent is filtered and the filter cake is washed with toluene (about 30 mL). The filtrate is concentrated in vacuo and the residue is dissolved in 50:50 toluene: heptane (about 17 mL) and the solution is then applied to a Silica Gel 60 column (43 g in 50:50 toluene: heptane). Column is eluted with 50:50 toluene:heptane (about 230 mL), toluene (about 430 mL), 10% EtOAc in toluene (about 240 mL) and 20% EtOAc in toluene (about 86 mL). Fractions of eluent containing pure compound are combined and concentrated in vacuo to provide the title compound as a syrup (5.79 g, 61%). ¹H NMR (CDCl₃) δ4.35 (m, 1H), 4.20 (m, 4H), 3.70 (s, 3H), 3.40 (m, 4H), 2.78 (m, 1H), 2.40-2.65 (m, 3H), 2.30 (m, 1H), 2.15 (m, 1H), 1.70-1.95 (m, 4H), 1.45-1.65 (m, 4H), 1.30 (m, 6H), 1.10 (m, 2H).

E. Preparation of 3S,4aR,6S,8aR Ethyl 6-(((2S)-2-(Ethoxycarbonyl)-4,4-difluoropyrrolidinyl)methyl)-1,2,3,4,4a,5,6,7,8,8a-decahydroisoquinoline-3-carboxylate (Free Base)

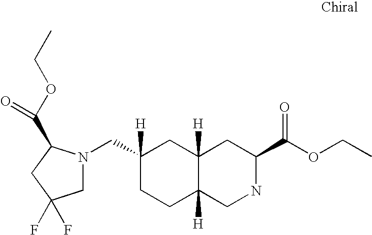

### Preparation of 3S,4aR,6S,8aR Ethyl 6-(((2S)-2-Ethoxycarbonyl)4,4-difluoropyrrolidinyl)methyl)-1,2,3,4,4a, 5,6,7,8,8a-decahydroisoquinoline-3-carboxylate.D-(−)-mandelic Acid Salt

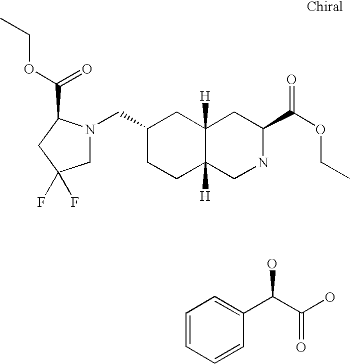

A solution of the compound of Step D above (9.7 g, 0.021 mol) in dichloromethane (about 70 mL) and toluene (about 30 mL) is cooled to 0-5° C. and treated with iodotrimethylsilane (12.7 g, 0.063 mol) dropwise. The reaction is allowed to warm to RT and is stirred for about 16 hours then is concentrated in vacuo. The residue is concentrated from MIBE (methyl t-butyl ether) several times then is dissolved in MTBE (about 70 mL) and toluene (about 30 mL) and is washed with saturated NaHCO₃ (about 100 mL), 1N sodium thiosulfate (about 100 mL), and water (about 100 mL). The organic layer is concentrated in vacuo and the residue is concentrated from MTBE several times and then is dissolved in toluene (about 22 mL) and then is treated with a solution of D-(−)-mandelic acid (2.75 g, 0.181 mol) in MTBE (about 33 mL). The mixture is stirred at RT for about 2 hours then is cooled to about −15° C. and stirred for about 2 hours. The D-(−)-mandelic acid salt is collected by filtration, washed with cold MTBE (about 73 mL at about −15 C.) and is dried to a crystalline solid (8.25 g, 71%). ¹H NMR (DMSOd₆) δ7.16-7.38 (m, 5H), 4.72 (s, 1H), 4.12 (m, 4H), 3.65 (m, 1H), 3.51 (m, 1H), 3.33 (m, 1H), 2.25-2.91 (m, 8H), 1.85 (m, 2H), 1.70 (m, 2H), 1.34-1.86 (m, 51), 1.15-1.38 (m, 7H), 0.81 (m, 1H).

### Example 2

### Preparation of 3S,4aR,6S,8aR Ethyl 6-(((2S)-2-(Ethoxycarbonyl)-4,4-difluoropyrrolidinyl)methyl)-1,2,3,4,4a,5,6,7,8,8a-decahydroisoquinoline-3-carboxylate.1,5-naphthalene Disulfonic Acid

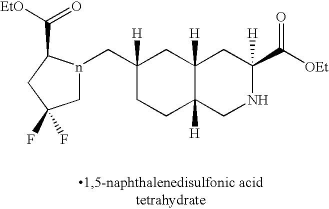

A solution of 1,5-naphthalenedisulfonic acid tetrahydrate (1.1 g, 3.05 mmol) in refluxing ethanol (about 5 mL) is treated with a solution of 3S,4aR,6S,8aR Ethyl 6(((2S)-2-(Ethoxycarbonyl)-4,4-difluoropyrrolidinyl)methyl)-1,2,3,4,4a,5,6,7,8,8a-decahydroisoquinoline-3-carboxylate (the free base diester compound from Example 1, Step E above) (1.2 g, 3.0 mmol) in ethanol (about 5 mL) and the mixture is heated at reflux until crystals began to form (about 5 minutes). Reaction is cooled and allowed to stand at RT for about 3 days. Product is collected by filtration, washed with ethanol (3×5 mL) and dried (2.0 g, 96%). Product can be recrystallized from water or methanol.

### EXAMPLE 3

### Preparation of 3S,4aR,6S,8aR 6-(((2S)-2-(Carboxylic acid)4,4-difluoropyrrolidinyl)methyl)-1,2,3,4,4a,5,6,7,8,8a-decahydroisoquinoline-3-carboxylic Acid.Dihydrochloride

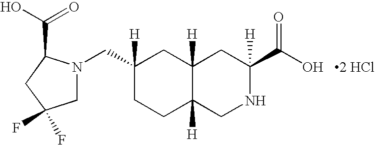

### Preparation of 3S,4aR,6S,8aR 6-(((2S)-2-(Carboxylic acid)-4,4-difluoropyrrolidinyl)methyl)-1,2,3,4,4a, 5,6,7,8,8a-decahydroisoquinoline-3-carboxylic Acid (Free Base)

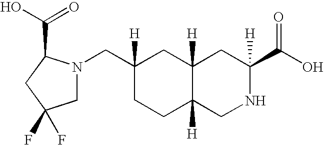

A mixture of the compound from Example 1, Step D above (16.3 g, 0.0353 mol) and 6M HCl (170 mL) is heated to reflux and the distillate from the reaction (about 15 mL) is collected over a period of 3 hours. The reaction is cooled and washed with dichloromethane (2×50 mL) then is treated with activated charcoal (about 8 g) and is then stirred at 60° C. for 30 min. The mixture is cooled and filtered through a pad of Hyflo. The Filter cake is washed with water (about 50 mL) and the aqueous filtrates are combined and concentrated in vacuo to a solid (14.2 g). This solid (8.61 g, 61% of the amount obtained) is concentrated from 2-propanol then is treated with 2-propanol (about 43 mL) and the mixture is heated at 50° C. for 1 hour. The mixture is cooled to about 0° C. and stirred for 30 min. Solid is collected by filtration, washed with cold 2-propanol (20 mL), and dried to a white powder (7.5 g), which is the dihydrochloride salt of the title compound. This powder (2.5 g, 33.3% of the amount obtained) is treated with 1N NaOH solution (11.9 mL) and the solution is then concentrated in vacuo. The residue is concentrated from EtOH then is treated with 50:50 EtOH:EtOAc. The mixture is warmed to about 35° C., then is cooled to RT and stirred for 1 hour, then is filtered. The filter cake is washed with 50:50 EtOH:EtOAc (5 mL) and the combined filtrates are concentrated to a foam. The foam is concentrated from EtOAc then is treated with EtOAc (10 mL) and stirred. The free base of the title compound is collected by filtration and is dried to a powder (1.96 g, potency corrected). Yield calculation for free base: 1.96 g divided by 0.333 divided by 0.61=9.65 g or 79%. ¹H NMR (D₂O+DCl) □4.55 (t, 1H), 4.07 (m, 1H), 3.83 (m, 1H), 3.65 (m, 1H), 3.25 (m, 1H), 2.95 (m, 4H), 2.59 (m, 1H), 1.89 (m, 3H), 1.77 (m, 1H), 1.65 (m, 1H), 1.46 (m, 3H), 1.35 (d, 1H), 1.20 (m, 1H), 0.84 (m, 1H).

### Example 4

To establish that the iGluR₅ receptor subtype is mediating neurogenic protein extravasation, a functional characteristic of migraine, the binding affinity of the panel compounds to the iGluR₅ receptor is first measured using standard methods. For example, the activity of compounds acting at the iGluR₅ receptor antagonists can be determined by radiolabelled ligand binding studies at the cloned and expressed human iGluR5 receptor (Korczak et al., 1994, Recept. Channels 3; 4149), and by whole cell voltage clamp electrophysiological recordings of currents in acutely isolated rat dorsal root ganglion neurons (Bleakman et al., 1996, Mol. Pharmacol. 49; 581-585). The selectivity of compounds acting at the iGluR₅ receptor subtype can then be determined by comparing antagonist activity at the iGluR₅ receptor with antagonist activity at other AMPA and kainate receptors. Methods useful for such comparison studies include: receptor-ligand binding studies and whole-cell voltage clamp electrophysiological recordings of functional activity at human GluR₁, GluR₂,GluR₃ and GluR₄ receptors (Fletcher et al., 1995, Recept. Channels 3; 21-31); receptor-ligand binding studies and whole-cell voltage clamp electrophysiological recordings of functional activity at human GluR₆ receptors (Hoo et al., Recept. Channels 2;327-338); and whole-cell voltage clamp electrophysiological recordings of functional activity at AMPA receptors in acutely isolated cerebellar Purkinje neurons (Bleakman et al., 1996, Mol. Pharmacol. 49; 581-585) and other tissues expressing AMPA receptors (Fletcher and Lodge, 1996, Pharmacol. Ther. 70; 65-89).

A. iGluR5 Antagonist Binding Affinity Profiles

Cell lines (HEK293 cells) stably transfected with human iGluR receptors are employed. Displacement of ³[H]AMPA by increasing concentrations of antagonist is measured on iGluR₁, iGluR₂, iGluR₃, and iGluR₄ expressing cells, while displacement of ³[H] kainate (KA) is measured on iGluR₅, iGluR₆, iGluR₇, and KA2-expressing cells. Estimated antagonist binding activity (Kᵢ) in μM, for example, is determined for Compounds of Formula I or Formula Ia. As an indicia of selectivity, the ratio of binding affinity to the iGluR₂ AMPA receptor subtype, versus the binding affinity to iGluR₅ kainate receptor subtype (Kᵢ at iGluR₂/Kᵢ at iGluR₅), is also determined. Compounds provided by the present invention display a greater binding affinity for iGluR₅ (lower Kᵢ) than that for iGluR₂, preferably at least 10 fold greater for iGluR₅ than that for iGluR₂, and more preferably at least 100 fold.

### Example 5

The following animal model may be employed to determine the ability of the compounds of Formula I or Formula Ia to inhibit protein extravasation, an exemplary functional assay of the neuronal mechanism of migraine.

### Animal Model of Dural Protein Extravasation

A. Harlan Sprague-Dawley rats (225-325 g) or guinea pigs from Charles River Laboratories (225-325 g) are anesthetized with sodium pentobarbital intraperitoneally (65 mg/kg or 45 mg/kg respectively) and are placed in a stereotaxic frame David Kopf Instruments) with the incisor bar set at −3.5 mm for rats or −4.0 mm for guinea pigs. Following a midline sagital scalp incision, two pairs of bilateral holes are drilled through the skull (6 mm posterially, 2.0 and 4.0 mm laterally in rats; 4 mm posteriorly and 3.2 and 5.2 mm laterally in guinea pigs, all coordinates referenced to bregma). Pairs of stainless steel stimulating electrodes, insulated except at the tips (Rhodes Medical Systems, Inc.), are lowered through the holes in both hemispheres to a depth of 9 mm (rats) or 10.5 mm (guinea pigs) from dura.

B. The femoral vein is exposed and a dose of the test compound is injected intravenously (i.v.) at a dosing volume of 1 ml/Kg or, in the alternative, test compound is administered orally (p.o) via gavage at a volume of 2.0 ml/Kg. Approximately 7 minutes post i.v. injection, a 50 mg/Kg dose of Evans Blue, a fluorescent dye, is also injected intravenously. The Evans Blue complexes with proteins in the blood and functions as a marker for protein extravasation. Exactly 10 minutes post-injection of the test compound, the left trigeminal ganglion is stimulated for 3 minutes at a current intensity of 1.0 mA (5 Hz, 4 msec duration) with a Model 273 potentiostat/galvanostat (EG&G Princeton Applied Research).

C. Fifteen minutes following stimulation, the animals are euthanized by exsanguination with 20 mL of saline. The top of the skull is removed to facilitate the collection of the dural membranes. The membrane samples are removed from both hemispheres, rinsed with water, and spread flat on microscopic slides. Once dried, the tissues are coverslipped with a 70% glycerol/water solution.

D. A fluorescence microscope (Zeiss) equipped with a grating monchromator and a spectrophotometer is used to quantify the amount of Evans Blue dye in each sample. An excitation wavelength of approximately 535 nm is utilized and the emission intensity at 600 nm is determined. The microscope is equipped with a motorized stage and also interfaced with a personal computer. This facilitates the computer-controlled movement of the stage with fluorescence measurements at 25 points (500 mm steps) on each dural sample. The mean and standard deviation of the measurements are determined by the computer.

E. The extravasation induced by the electrical stimulation of the trigeminal ganglion has an ipsilateral effect (i.e. occurs only on the side of the dura in which the trigeminal ganglion is stimulated). This allows the other (unstimulated) half of the dura to be used as a control. The ratio (“extravasation ratio”) of the amount of extravasation in the dura from the stimulated side, over the amount of extravasation in the unstimulated side, is calculated. Control animals dosed with only with saline, yield an extravasation ratio of approximately 2.0 in rats and apprximately 1.8 in guinea pigs. In contrast, a compound which completely prevents the extravasation in the dura from the stimulated side yields an extravasation ratio of approximately 1.0.

F. Dose-response curves may be generated for each of the compounds of Formula I and Formula Ia and the dose that inhibits the extravasation by 50% (ID₅₀) or 100% (ID₁₀₀) can then be approximated. The compound 3S,4aR,6S,8aR Ethyl 6-(((2S)-2-Ethoxycarbonyl)4,4-difluoropyrrolidinyl)methyl)-1,2,3,4,4a,5,6,7,8,8a-decahydroisoquinoline-3-carboxylate.dihydrochloride provides an ID₁₀₀ of approximately 0.01 ng/Kg when administerd orally to rats.

What is claimed is:

1. A pharmaceutically acceptable salt of a compound of the formula: 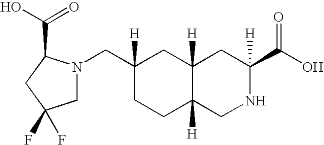
or a prodrug thereof, wherein the pharmaceutically acceptable salt is selected from the group consisting of the d-(−)-mandelic acid salt or the 1,5-naphthalene disulfonic acid salt.

2. The pharmaceutically acceptable salt according to claim 1, wherein the salt is the D-(−)-mandelic acid salt.

3. The pharmaceutically acceptable salt according to claim 1, wherein the salt is the 1,5-naphthalene disulfonic acid salt.

4. A compound which is 3S,4aR,6S,8aR 6-(((2S)-2-(carboxylic acid)-4,4-difluoropyrrolidinyl)methyl)-1,2,3,4,4a,5,6,7,8,8a-decahydroisoquinoline-3-carboxylic acid.D-(−)-mandelic acid.

5. A compound which is 3S,4aR,6S,8aR 6-(((2S)-2-(carboxylic acid)-4,4-difluoropyrrolidinyl)methyl)-1,2,3,4,4a,5,6,7,8,8a-decahydroisoquinoline-3carboxylic acid.1,5-naphthalene disulfonic acid.

6. A pharmaceutically acceptable salt of a compound of the formula: 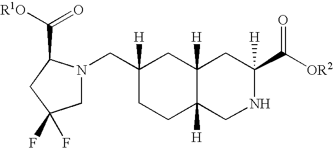
wherein
R¹ and R² each independently represent hydrogen, (C₁-C₂₀)alkyl, (C₂-C₆)alkenyl, (C₁-C₆)alkylaryl, (C₁-C₆)alkyl(C₃-C₁₀)cycloalkyl, (C₁-C₆)alkyl-N,N-C₁-C₆ dialkylamine, (C₁-C₆)alkyl-pyrrolidine, (C₁-C₆)alkyl-piperidine, or (C₁-C₆)alkyl-morpholine, with the proviso that at least one of R¹ and R² are other than hydrogen;
wherein the pharmaceutically acceptable salt is selected from the group consisting of the D-(−)-mandelic acid salt or the 1,5-naphthalene disulfonic acid salt.

7. The pharmaceutically acceptable salt according to claim 6, wherein R¹ and R² are each independently (C₁-C₂₀)alkyl.

8. The pharmaceutically acceptable salt according to claim 7, wherein R¹ and R² are each independently (C₁-C₆)alkyl.

9. The pharmaceutically acceptable salt according to claim 8, wherein the salt is the D-(−)-mandelic acid salt.

10. The pharmaceutically acceptable salt according to claim 8, wherein the salt is the 1,5-naphthalene disulfonic acid salt.

11. A compound which is 3S,4aR,6S,8aR Ethyl 6-(((2S)-2-(Ethoxycarbonyl)-4,4-difluoropyrrolidinyl)methyl)-1,2,3,4,4a,5,6,7,8,8a-decahydroisoquinoline-3-carboxylate.D-(−)-mandelic acid.

12. A compound which is 3S,4aR,6S,8aR Ethyl 6-(((2S)-2-(Ethoxycarbonyl)-4,4-difluoropyrrolidinyl)methyl)-1,2,3,4,4a,5,6,7,8,8a-decahydroisoquinoline-3-carboxylate.1,5-naphthalene disulfonic acid.

13. A method of treating a neurological disorder or neurodegenerative disease comprising administering to a patient in need thereof, an effective amount of a pharmaceutically acceptable salt according to claim 1.

14. The method according to claim 13, wherein the neurological disorder is migraine.

15. The method according to claim 14, wherein the pharmaceutically acceptable salt is 3S,4aR,6S,8aR 6-(((2S)-2-(carboxylic acid)-4,4-difluoropyrrolidinyl)methyl)-1,2,3,4,4a,5,6,7,8,8a-decahydroisoquinoline-3-carboxylic acid.D-(−)-mandelic acid.

16. The method according to claim 14, wherein the pharmaceutically acceptable salt is 3S,4aR,6S,8aR Ethyl 6-(((2S)-2-(Ethoxycarbonyl)-4,4-difluoropyrrolidinyl)methyl)-1,2,3,4,4a,5,6,7,8,8a-decahydroisoquinoline-3-carboxylate.1,5-naphthalene disulfonic acid.

17. A method of treating a neurological disorder or neurodegenerative disease comprising administering to a patient in need thereof, an effective amount of a pharmaceutically acceptable salt according to claim 6.

18. The method according to claim 17, wherein the neurological disorder is migraine.

19. The method according to claim 17, wherein the pharmaceutically acceptable salt is 3S,4aR,6S,8aR 6-(((2S)-2-(carboxylic acid)-4,4-difluoropyrrolidinyl)methyl)-1,2,3,4,4a,5,6,7,8,8a-decahydroisoquinoline-3-carboxylic acid.D-(−)-mandelic acid.

20. The method according to claim 17, wherein the pharmaceutically acceptable salt is 3S,4aR,6S,8aR Ethyl 6-(((2S)-2-(Ethoxycarbonyl)-4,4-difluoropyrrolidinyl)methyl)-1,2,3,4,4a,5,6,7,8,8a-decahydroisoquinoline-3carboxylate.1,5-naphthalene disulfonic acid.

21. A pharmaceutical composition comprising an effective amount of the pharmaceutically acceptable salt according to claim 1, in combination with a pharmaceutically acceptable carrier, diluent, or excipient.

22. A pharmaceutical composition comprising an effective amount of the pharmaceutically acceptable salt according to claim 6, in combination with a pharmaceutically acceptable carrier, diluent, or excipient.

23. The use of a compound according to claim 1 for the manufacture of a medicament for the treatment of migraine.

24. The use of a compound according to claim 6 for the manufacture of a medicament for the treatment of migraine.

25. The use of a compound according to claim 1 for the treatment of migraine.

26. The use of a compound according to claim 6 for the treatment of migraine.

27. A process for preparing a compound of the formula: 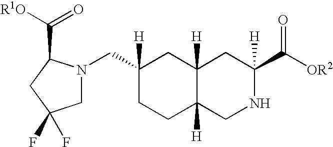
wherein
R¹ and R² each independently represent hydrogen, (C₁-C₂₀)alkyl, (C₂-C₆)alkenyl, (C₁-C₆)alkylaryl, (C₁-C₆)alkyl(C₃-C₁₀)cycloalkyl, (C₁-C₆)alkyl-N,N-C₁-C₆ dialkylamine, (C₁-C₆)alkyl-pyrrolidine, (C₁-C₆)alkyl-piperidine, or (C₁-C₆)alkyl-morpholine, with the proviso that at least one of R¹ and R² are other than hydrogen,
comprising combining a compound of structure (2) 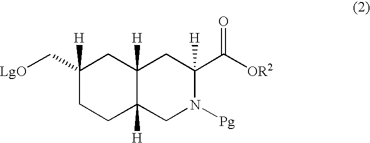
wherein R² is as defined above, Pg is a suitable nitrogen protecting group, and LgO is a suitable leaving group, with a suitable base in a suitable solvent, followed by addition of a compound of structure (3) 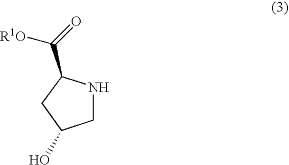
wherein R¹ is as defined above, followed by oxidation to a compound of structure (5) 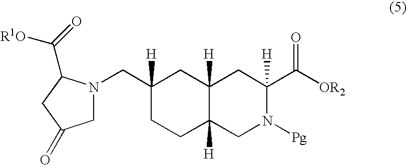
followed by halogenation and removal of the nitrogen protecting group.
